SEQUENCE LISTING (23 sequences)

SEQ ID NO: 1 - DNA, 40 bp, Artificial Sequence
  primer
ctaggcgccggaattagatctaccatggagacagacagac

SEQ ID NO: 2 - DNA, 36 bp, Artificial Sequence
  primer
gtttgttgcgccggatccacactctcccctgttgaa

SEQ ID NO: 3 - DNA, 36 bp, Artificial Sequence
  primer
ttcaacaggggagagtgtggatccggcgcaacaaac

SEQ ID NO: 4 - DNA, 36 bp, Artificial Sequence
  primer
gctgagcctgatgtccatgggtccaggattctcttc

SEQ ID NO: 5 - DNA, 36 bp, Artificial Sequence
  primer
gaagagaatcctggacccatggacatcaggctcagc

SEQ ID NO: 6 - DNA, 36 bp, Artificial Sequence
  primer
ggatggaggaggctcgagtttacccggagacaggga

SEQ ID NO: 7 - DNA, 36 bp, Artificial Sequence
  primer
tccctgtctccgggtaaactcgagcctcctccatcc

SEQ ID NO: 8 - DNA, 39 bp, Artificial Sequence
  primer
ttcgttaacctcgagagatcttcacgctagagaatgagg

SEQ ID NO: 9 - DNA, 699 bp, Artificial Sequence
  light chain
atggagacagacagactcctgctatgggtgctgctgctctgggtgccaggctccactggtgacactgtactgacccagtctcctgctttggctgtgtctccaggagagagggttaccatctcctgtagggccagtgacagtgtcagtacacttatgcactggtaccaacagaaaccaggacagcaacccaaactcctcatctatctagcatcacacctagaatctggggtccctgccaggttcagtggcagtgggtctgggacagacttcaccctcaccattgatcctgtggaggctgatgacactgcaacctattactgtcagcagagttggaatgatccgtggacgttcggtggaggcaccaagctggaattgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - DNA, 66 bp, Artificial Sequence
  P2A
ggatccggcgcaacaaacttctctctgctgaaacaagccggagatgtcgaagagaatcctggaccc

SEQ ID NO: 11 - DNA, 1383 bp, Artificial Sequence
  heavy chain
atggacatcaggctcagcttggttttccttgtccttttcataaaaggtgtccagtgtgaggtgcagctggtggagtctgatggaggcttagtgcagcctggaaggtccctaaaactcccctgtgcagcctcaggattcactttcagtgactattacatggcctgggtccgccaggctccaacgaaggggctggagtgggtcgcaagcattagttatgatggtagtagcacttactatcgagactccgtgaagggccgattcactatctccagagataatgcaaaaagcaccctatacctgcaaatggacagtctgaggtctgaggacacggccacttattactgcggaagacacagtagctactttgattactggggccaaggagtcatggtcacagtctcgagcgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - DNA, 228 bp, Artificial Sequence
  Transmembrane domain
ctcgagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtga

SEQ ID NO: 13 - protein, 791 aa, Artificial Sequence
  antibody
METDRLLLWVLLLWVPGSTGDTVLTQSPALAVSPGERVTISCRASDSVSTLMHWYQQKPGQQPKLLIYLASHLESGVPARFSGSGSGTDFTLTIDPVEADDTATYYCQQSWNDPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGATNFSLLKQAGDVEENPGPMDIRLSLVFLVLFIKGVQCEVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKLEPPPSTVSNMATVAVLVVLGAAIVTGAVVAFVMKMRRRNTGGKGGDYALAPGSQTSDLSLPDCKVMVHDPHSLA

SEQ ID NO: 14 - protein, 216 aa, Artificial Sequence
  Fc
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 109 aa, Artificial Sequence
  CH2
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 16 - DNA, 708 bp, Artificial Sequence
  light chain
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgttagcggcgactacttagcctggtaccagcagaaacttggccaggctcccaggctcctcatctatggtgcatccatcagggccactggcatcccagacaggttcagtggcagtgggtctgggacagacttcactctcaccatcagcaaactggagcctgaagattttgcagtgtattactgtcagcagtatggtagctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 17 - DNA, 1392 bp, Artificial Sequence
  heavy chain
atgaaacacctgtggttcttcctcctcctggtggcagctcccagatgggtcctgtctcaggtgcagctgcaggagtcgggcccaggactggtgaagccttcggggaccctgtcactcacctgcgctgtctctggtggctccatcagcagtagtatctggtggagctgggtccgccagcccccagggaaggggctggaatggattggggaaatccatcatagtgggaacaccaactgcaatccgtccctcaagagtcgagtcaccatatcaatagacaagtccaagaaccagttctccctgaaactgacctctgtgaccgccgcggacacggccgtatattattgtgcgagagcgggaactggaactacgcttgactactggggccagggaaccctggtcaccgtctcgagcgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 235 aa, Artificial Sequence
  light chain
METPAQLLFLLLLWLPDTTGEIVLTQSPGTLSLSPGERATLSCRASQSVSGDYLAWYQQKLGQAPRLLIYGASIRATGIPDRFSGSGSGTDFTLTISKLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 463 aa, Artificial Sequence
  heavy chain
MKHLWFFLLLVAAPRWVLSQVQLQESGPGLVKPSGTLSLTCAVSGGSISSSIWWSWVRQPPGKGLEWIGEIHHSGNTNCNPSLKSRVTISIDKSKNQFSLKLTSVTAADTAVYYCARAGTGTTLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 99 aa, Artificial Sequence
  fragment of Fc
PVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPI

SEQ ID NO: 21 - DNA, 39 bp, Artificial Sequence
  primer
  misc_feature (19)..(20): n is a, c, g, or t
ccaccgtgcccagcaccannsgtggcaggaccgtcagtc

SEQ ID NO: 22 - DNA, 44 bp, Artificial Sequence
  primer
tgcaaggtctccaacaaaggctggccagcccccatcgagaaaac

SEQ ID NO: 23 - DNA, 54 bp, Artificial Sequence
  primer
  misc_feature (20)..(20): n is a, c, g, or t
acgtgcgtggtggtggackngwbgvasrwssacchggaggtccagttcaactgg

(19) United States
(12) Patent Application Publication
(10) Pub. No.: US 2023/0220042 A1
(43) Pub. Date: Jul. 13, 2023

(54) ANTIBODY FC REGION HAVING ENHANCED BINDING AFFINITY TO FCYRIIB

(71) Applicant: SHANGHAI JIAO TONG UNIVERSITY SCHOOL OF MEDICINE, Shanghai (CN)

(72) Inventors: Fubin LI, Shanghai (CN); Yan ZHANG, Shanghai (CN); Mi ZHANG, Shanghai (CN); Yanxia BI, Shanghai (CN); Shihao TIAN, Shangha (CN); Huihui ZHANG, Shanghai (CN); Shujun LIU, Shanghai (CN)

(73) Assignee: SHANGHAI JIAO TONG UNIVERSITY SCHOOL OF MEDICINE, Shanghai (CN)

(21) Appl. No.: 17/756,836

(22) PCT Filed: Dec. 3, 2020
(86) PCT No.: PCT/CN2020/133685

§ 371 Date: Jun. 3, 2022

(30) Foreign Application Priority Data
Dec. 3, 2019 (CN) 201911223227.1

(51) Int. Cl. C07K 14/735; C12N 15/63; C07K 16/28; A61P 35/00

(52) U.S. Cl. CPC C07K 14/70535; C12N 15/63; C07K 16/2878; A61P 35/00

(57) ABSTRACT

Provided is a modified Fc region. The Fc region has at least one amino acid mutation with respect to a parent Fc region. The modified Fc region has improved affinity to FcγRIIB with respect to the affinity of the parent Fc region to FcγRIIB Also provided is an antibody containing the modified Fc region, especially an agonistic antibody. The modified Fc region has significant use for optimizing the agonistic activity of the antibody.

### FIELD

The present disclosure belongs to the field of biopharmaceuticals, and In one embodiment relates to a series of variants of Fc regions of human IgG2 antibodies. These variants have enhanced binding affinity to FcγRIIB Agonistic antibodies comprising these Fc region variants are expected to have better agonistic activity.

### BACKGROUND OF DISCLOSURE

The development of biomacromolecule drugs provides new methods and possibilities for the treatment of a variety of diseases, in particular, molecular targeted therapy based on antibody and heavy chain constant regions (including Fc regions), including antibodies and heavy chain constant region fusion proteins, which have gained great success in the field of biopharmaceuticals and continue to become the focus of this field.

According to different functions, the biomacromolecules can be mainly classified into three categories: effector molecules that clear targets (molecules and cells), blocking molecules that block target-involved signaling pathways, and agonistic molecules that activate signaling pathways downstream of the target. Tumor immunotherapy has gained important breakthroughs in recent years. This benefits from the use of antibodies that increase the activity of immune cells to kill tumors by blocking inhibitory immune checkpoint molecules. However, there are still a large number of cancer patients not responding to existing treatment means. Therefore, on the one hand, it is necessary to optimize the existing tumor immunotherapy means; on the other hand, new tumor immunotherapy drugs need to be developed. It should be noted that there is a class of tumor immunotherapy means referred to as “Agonistic antibodies”, which are capable of enhancing the anti-tumor immune response to indirectly kill tumor cells by binding target molecules on the immune cell surface that pass the immune activation signal and activate important immune activation signal pathways controlled by the target molecules. However, while agonistic tumor immunotherapy antibodies have demonstrated their enormous potential in animal models, and have been widely accepted and welcomed as a good tumor immunotherapy concept. However, the research and development of such antibodies have not yet been successful and are a major challenge in the field of tumor immunotherapy. In addition, activation by agonistic antibodies is also an advantageous means of intervention and regulation of key signal pathways of other biological processes, and has a broad application prospect in the field of disease prevention and treatment. For example, activating an immunosuppressive signal pathway may be advantageous for mitigating inflammation and autoimmune symptoms.

An antibody mainly interacts with FcγR through its constant region, and the binding affinity of different antibodies to FcγR also varies, which also affects the function of the antibody in vivo. Antibody Fc modification is an important means and trend to optimize the therapeutic antibody, and altering the interaction affinity of the Fc region of the antibody to the key protein such as FcγR is a very effective way to optimize the activity of the antibody.

### SUMMARY OF DISCLOSURE

For the above problems, the present disclosure provides amino acid mutations and combinations capable of enhancing the affinity of Fc to FcγRIIB by performing mutation modification on the Fc region based on IgG; and the Fc containing these amino acid mutations can be used to optimize the activity of the antibody, in particular, to improve the activation activity of the agonistic antibody.

In one embodiment of the present disclosure, provided herein is a mutant Fc polypeptide fragment having the following features:

(i) said mutant Fc polypeptide fragment has a mutation with respect to a corresponding wild-type Fc fragment before mutation; and

(ii) the affinity of the mutant Fc region to FcγRIIB is increased compared to the wild-type Fc region; and the wild-type Fc is the Fc of wild-type IgG2.

In another embodiment, the mutant Fc polypeptide fragment has a mutation selected from the group consisting of: L328, H268, 5267, P271, G327, or a combination thereof, with respect to a corresponding wild-type Fc fragment before mutation;

and, the numbering of all amino acids is based on the IgG Eu numbering.

In another embodiment, the affinity may be embodied as a screening enrichment degree or a binding analysis signal.

In another embodiment, the wild-type Fc fragment is residue at 233th to residue at 332th of the amino acid sequence of the IgG2 according to the IgG Eu numbering, and the amino acid sequence of the wild-type Fc fragment is as shown in SEQ ID NO: 20.

In another embodiment, the mutant Fc polypeptide fragment has an enhanced affinity to FcγRIIB as compared to a wild-type Fc fragment.

In another embodiment, the affinity of the mutant Fc polypeptide fragment to FcγRIIB is increased by more than 1 times higher than the affinity of the wild-type Fc fragment to FcγRIIB; preferably, 2, 3, 4, 5, 10, 20, 50, or 100 times higher.

In another embodiment, the affinity of the mutant Fc polypeptide fragment to the activating FcγRs is lower than that to FcγRIIB

In another embodiment, the affinity of the mutant Fc polypeptide fragment to the FcγRIIB is increased and the affinity of the mutant Fc polypeptide fragment to the activating FcγRs is reduced (I/A ratio is higher than that of the wild-type Fc fragment), as compared to the wild-type Fc fragment.

In another embodiment, the ratio (RIIB/RIIAR) of the affinity of the mutant Fc polypeptide fragment to the FcγRIIB and FcγRIIA^{131R} is higher than that of wild type IgG2.

In another embodiment, the activating FcγRs includes: FcγRI, FcγRIIA^{131H}, FcγRIIA^{131R}, FcγRIIIA^{158F} and FcγRIIIA^{158V}.

In another embodiment, the mutation L328 includes L328W, L328E, L328Y, L328F, L328M, L328A, L328G, L328N, L328P, L328R, L328V; preferably L328W or L328E.

In another embodiment, the mutation H268 includes H268D, H268S, H268E, H268K, H268N, preferably is H268D, H268S, or H268E.

In another embodiment, the mutation S267 includes S267E, S267V, S267D, S267M, S267Q, S267A, S267G, S267R, S267N, S267W; preferably S267E, S267V, S267D, S267M; more preferably is S267E.

In another embodiment, the mutation P271 includes P271C, P271G, P271V, P271A, P271W, P271Y, P271Q, P271T, P271I, P271L, P271S; preferably is P271C, P271G, P271V, P271A, P271W, P271Y, P271Q, P271T; more preferably is P271C, P271G.

In another embodiment, the mutation G327 includes G327A, G327L, G327S; preferably G327A.

In another embodiment, the mutant Fc polypeptide fragment has a mutation selected from the group consisting of L328W, L328E, H268D, H268S, H268E, S267E, A330S, P233G, P271G, P271C, P271V, P271A, P271W, P271Y, G327A, or a combination thereof, with respect to the wild-type Fc fragment; and the numbering of all amino acids is based on the IgG Eu numbering.

In another embodiment, the mutant Fc polypeptide fragment has a mutation in at least one of L328 and H268 with respect to the wild-type Fc fragment, and the specific mutations in the mutant Fc polypeptide fragment are selected from one of the group consisting of: L328W, L328E, H268D, H268S, H268E, H268D/S298L/L328W, S267V/S298L/L328W, V234M/S267E/S298L/L328W, A235W/V266L/S298L/L328W, V234Q/A235G/P238L/S239V/H268D/G327A/L328E/A330S/1332T, S239V/V266L/S298L/L328W, V266L/L328W, V266L/S267D/H268D/E269D/P271Q, S267E/H268S/E269D, P233F/V266L/S298L/L328W, V266L/S298L/L328W, V234Q/A235G/P238L/S239V/G327A/L328E/A330S/I332T, V266L/S267E/H268S/E269V, V266L/S267E/H268S/E269V/P271C, V266L/S267E/H268S/E269A/P271C, S267V/S298G/L328W, V266L/S267M/H268E/P271Q, V266L/S267E/H268S/E269G/P271T, V234Q/V266L/S267D/H268D/E269D/P271Q, V234Q/A235G/P238L/S239V/S267E/H268S/E269D, V266L/S267E/H268S/P271V, V234Q/A235G/P238L/S239V/S267E/S298L/G327A/L328E/A330S/I332T, V234Q/V266L/S267E/H268S/E269V, L328E/I332T; and the numbering of all amino acids is based on the IgG Eu numbering.

In another embodiment, the mutant Fc polypeptide fragment has a mutation at S267 with respect to the wild-type Fc fragment, and the specific mutation in the mutant Fc polypeptide fragment is selected from one of the group consisting of: S267E/A330S, S267E/S298G, P233G/S267E, V234M/S267E/S298G/I332L, P233G/S267E/S298G, V266L/S267E/E269K/P271G, P238Q/S267E, S267A/P271C, S267A/P271G, S267E/D270H, S267E/P271C, S267E/P271V, S267E/P271W, S267E/P271Y, S267E/S298R, S267M/P271C, S267M/P271G, S267V/S298L, S267E/P329R; and the numbering of all amino acids is based on the IgG Eu numbering.

In another embodiment, the mutant Fc polypeptide fragment has a mutation at S267 with respect to the wild-type Fc fragment, and the specific mutation in the mutant Fc polypeptide fragment is selected from one of the group consisting of: S267E/A330S, S267E/S298G, P233G/S267E, V234M/S267E/S298G/I332L, P233G/S267E/S298G, V266L/S267E/E269K/P271G, S267E/P271C; and the numbering of all amino acids is based on the IgG Eu numbering.

In another embodiment, the mutant Fc polypeptide fragment has a mutation at P271 with respect to the wild-type Fc fragment, and the specific mutation in the mutant Fc polypeptide fragment is selected from one of the group consisting of: V266L/S267E/E269K/P271G, V266A/P271C, V266A/P271G, S267A/P271C, S267A/P271G, S267E/P271C, S267E/P271V, S267E/P271W, S267E/P271Y, S267M/P271C, S267M/P271G, V266G/P271C, P271A/S298R, P271G/G236V, P271G/P329S, P271G/P331C, P271G/P331T, P271G/S298D, P271G/S298E, P271G/S298G, P271G/S298K, P271G/S298L, P271G/S298N, P271G/S298R, P271G/T299A, P271G/T299M, P271G/T299S, P271G/T299W, P271G/I332L; and the numbering of all amino acids is based on the IgG Eu numbering.

In another embodiment, the mutant Fc polypeptide fragment has a mutation at G327 with respect to the wild-type Fc fragment, and the specific mutation in the mutant Fc polypeptide fragment is selected from one of the group consisting of: G327A/A330R, G327A/A330V, G327A/I332A, G327A/I332C, G327A/I332E.

In another embodiment, the specific mutation of the mutant Fc polypeptide fragment with respect to the wild-type Fc fragment is selected from one of the group consisting of: H268D/S298L/L328W, S267V/S298L/L328W, V234M/S267E/S298L/L328W, A235W/V266L/S298L/L328W, V234Q/A235G/P238L/S239V/H268D/G327A/L328E/A330S/I332T; and the numbering of all amino acids is based on the IgG Eu numbering.

In one embodiment of the present disclosure, provided herein is a mutant immunoglobulin Fc region comprising the mutant Fc polypeptide fragment according to embodiments of the present disclosure.

In another embodiment, the immunoglobulin is human IgG2.

In another embodiment, the mutant immunoglobulin Fc region has an enhanced affinity to FcγRIIB as compared to a wild-type IgG2 Fc region.

In another embodiment, the affinity of the mutant immunoglobulin Fc region to FcγRIIB is increased by more than 1 times higher than the affinity of the wild-type IgG2 Fc region to FcγRIIB; preferably, 2, 3, 4, 5, 10, 20, 50, or 100 times higher.

In another embodiment, the affinity may be embodied as a screening enrichment degree or a binding analysis signal.

In another embodiment, the affinity of the mutant immunoglobulin Fc region to an activating FcγR is lower than that to FcγRIIB

In another embodiment, the affinity of the mutant immunoglobulin Fc region to the FcγRIIB is increased and the affinity of the mutant immunoglobulin Fc region to the activating FcγR is reduced (I/A ratio is higher than that of the wild-type IgG2 Fc region), as compared to the wild-type IgG2 Fc region.

In another embodiment, the ratio (RIIB/RIIAR) of the affinity of the mutant immunoglobulin Fc region to the FcγRIIB and FcγRIIA^{131R} is higher than that of wild type IgG2.

In one embodiment of the present disclosure, provided herein is an antibody comprising the mutant Fc polypeptide fragment according to embodiments of the present disclosure or the mutant immunoglobulin Fc region according to embodiments of the present disclosure.

In another embodiment, the antibody is an antibody based on a human IgG2 backbone.

In another embodiment, the antibody is an agonistic antibody.

In another embodiment, the antibody In one embodiment targets a tumor necrosis factor receptor superfamily.

In another embodiment, the antibody is capable of In one embodiment binding to a target selected from the group consisting of CD40, DR5, OX40, CD137, CD27, CD30, GITR, HVEM, TACI, DR4, FAS, or a combination thereof.

In another embodiment, the antibody is capable of In one embodiment binding to OX40.

In another embodiment, the antigen targeted by the antibody is an immune receptor molecule, or a combination thereof.

In another embodiment, the antigen targeted by the antibody is an immunosuppressive receptor molecule, and the immunosuppressive receptor molecule is selected from the group consisting of: PD-1, CTLA-4, VISTA, TIM-3, BTLA, LAG-3, or a combination thereof.

In another embodiment, the antibody is a human antibody, a humanized antibody, or a chimeric antibody.

In another embodiment, the antibody is a monoclonal antibody or a polyclonal antibody, preferably a monoclonal antibody.

In one embodiment of the present disclosure, provided herein is a fusion protein comprising the mutant Fc polypeptide fragment according to embodiments of the present disclosure, the mutant immunoglobulin Fc region according to embodiments of the present disclosure, or the antibody according to embodiments of the present disclosure.

In another embodiment, the fusion protein further includes other protein sequences having receptor agonist functions or fragments thereof.

In another embodiment, the other protein having a receptor agonist function is a cytokine of the TNF gene family.

In another embodiment, the other protein having a receptor agonist function includes a ligand molecule of an immune receptor.

In another embodiment, the other protein having the receptor agonist function includes a ligand molecule of an immune receptor selected from any of the group consisting of: CD80, CD86, ICOSL, OX40L, CD137L, CD40L, CD30L, CD27L, CD244, CD150, CD48, CD84, CD319, Ly118 or CD229, or a combination thereof.

In another embodiment, the other protein having the receptor agonist function includes a ligand molecule of an immune receptor, and the ligand molecule of the immune receptor is selected from the group consisting of: PD-L1, PD-L2, B7-H3, B7-H4, CD47, VISTA, HVEM, GALS, or a combination thereof.

In another embodiment, the fusion protein may further include a tag sequence useful in expression and/or purification; preferably, the tag sequence includes a 6His tag, an HA tag, and/or a FLAG tag.

In one embodiment of the present disclosure, provided herein is an isolated polynucleotide encoding the mutant Fc polypeptide fragment according to embodiments of the present disclosure, the mutant immunoglobulin Fc region according to embodiments of the present disclosure, the antibody according to embodiments of the present disclosure, or the recombinant protein according to embodiments of the present disclosure.

Embodiments of the present disclosure provides a vector containing the isolated polynucleotide according to embodiments of the present disclosure.

In another embodiment, the vector is selected from the group consisting of DNAs, RNAs, viral vectors, plasmids, transposons, other gene transfer systems, or combinations thereof.

In another embodiment, the vector includes a viral vector, such as a vector of a lentivirus, an adenovirus, an AAV virus, a retrovirus, or a combination thereof.

Embodiments of the present disclosure provides a host cell containing the vector according to embodiments of the present disclosure, or having the polynucleotide according to embodiments of the present disclosure integrated into the genome of the cell;

or, the host cell expresses the mutant Fc polypeptide fragment according to embodiments of the present disclosure, the mutant immunoglobulin Fc region according to embodiments of the present disclosure, the antibody according to embodiments of the present disclosure, or the recombinant protein according to embodiments of the present disclosure.

In another embodiment, the host cell includes a prokaryotic cell or a eukaryotic cell.

In another embodiment, the host cell is selected from the group consisting of: E coli, yeast cells, insect cells, avian cells and mammalian cells.

In one embodiment of the present disclosure, provided herein is a pharmaceutical composition comprising:

(a) the mutant Fc polypeptide fragment according to embodiments of the present disclosure, the mutant immunoglobulin Fc region according to embodiments of the present disclosure, the antibody according to embodiments of the present disclosure, or the recombinant protein according to embodiments of the present disclosure; and

(b) a pharmaceutically acceptable carrier.

In another embodiment, the pharmaceutical composition may further include other drugs for treating tumors, such as cytotoxic drugs.

In another embodiment, the pharmaceutical composition may further include other active substances having a receptor agonist function.

In another embodiment, the pharmaceutical composition is an injection dosage form.

In one embodiment of the present disclosure, provided herein is a method of producing the mutant Fc polypeptide fragment according to embodiments of the present disclosure, the mutant immunoglobulin Fc region according to embodiments of the present disclosure, the antibody according to embodiments of the present disclosure, or the recombinant protein according to embodiments of the present disclosure, comprising the steps of:

(i) culturing the host cell according to embodiments of the disclosure under suitable conditions to obtain a culture comprising said mutant Fc polypeptide fragment, said mutant immunoglobulin Fc region, said antibody, or said recombinant protein; and

(ii) purifying and/or separating said mutant Fc polypeptide fragment, said mutant immunoglobulin Fc region, said antibody, or said recombinant protein from the culture obtained in step (i).

In another embodiment, the purification can be performed by protein A affinity column purification to obtain a target antibody.

In another embodiment, the purification can be performed by protein G affinity column purification to obtain a target antibody.

In another embodiment, the purity of the target antibody after purification and/or separation is greater than 95%, greater than 96%, greater than 97%, greater than 98%, greater than 99%, preferably 100%.

In one embodiment of the present disclosure, provided herein is use of the mutant Fc polypeptide fragment according to embodiments of the present disclosure, the mutant immunoglobulin Fc region according to embodiments of the present disclosure, the antibody according to embodiments of the present disclosure, the recombinant protein according to embodiments of the present disclosure, the polynucleotide according to embodiments of the present disclosure, the vector according to embodiments of the present disclosure and/or the host cell according to embodiments of the present disclosure, in the manufacture of a pharmaceutical composition for tumor immunotherapy, reducing inflammation and/or reducing autoimmune symptoms.

In one embodiment of the present disclosure, provided herein is a method of treating a disease, comprising the steps of: administering to a subject in need thereof the mutant Fc polypeptide fragment according to embodiments of the present disclosure, the mutant immunoglobulin Fc region according to embodiments of the present disclosure, the antibody according to embodiments of the present disclosure, the recombinant protein according to embodiments of the present disclosure, the polynucleotide according to embodiments of the present disclosure, the vector according to embodiments of the present disclosure, the host cell according to embodiments of the present disclosure, and/or the pharmaceutical composition according to embodiments of the present disclosure.

In another embodiment, the subject includes a mammal, preferably is a human.

In another embodiment, the disease is selected from the group consisting of: tumors, inflammatory diseases, autoimmune diseases, or combinations thereof.

In another embodiment, the cancer includes, but is not limited to, breast cancer, prostate cancer, lung cancer, ovarian cancer, cervical cancer, skin cancer, melanoma, colon cancer, gastric cancer, liver cancer, esophageal cancer, renal cancer, throat cancer, thyroid cancer, pancreatic cancer, testicular cancer, brain cancer, bone cancer and blood cancer (such as leukemia, chronic lymphocytic leukemia), etc.

In another embodiment, the cancer includes, but is not limited to, basal cell carcinoma, biliary cancer, bladder cancer, bone cancer, brain and central nervous system (CNS) cancer, cervical cancer, chorionic carcinoma, colorectal cancer, connective tissue cancer, digestive system cancer, endometrial cancer, esophageal cancer, ocular cancer, head and neck cancer, gastric cancer, epithelial neoplasm, renal cancer, throat cancer, liver cancer, lung cancer (small cell, large cell), lymphoma (including Hodgkin lymphoma and non-Hodgkin lymphoma); melanoma; neuroblastoma; oral cancer (e.g., lip, tongue, mouth and pharyngeal); ovarian cancer; pancreatic cancer; retinoblastoma; rhabdomyosarcoma; rectal cancer; respiratory cancer; sarcoma; skin cancer; gastric cancer; testicular cancer; thyroid cancer; uterine cancer; urinary system cancer; and other cancers and sarcoma.

In another embodiment, the method can be used to stimulate an immune response to treat tumors by inhibiting or delaying the growth of a tumor or reducing the size of a tumor.

In another embodiment of the present disclosure, further provided is a method for screening amino acid mutations enhancing protein-molecular binding affinity, the method includes the following steps:

1) providing a parent protein sequence, introducing amino acid mutations in the parent protein sequence by PCR;

2) constructing the parent protein and the mutated protein into an expression vector to form a mutation library;

3) transfecting the expression vector into mammalian cells, and expressing the parent protein and the mutated protein on the cell surface;

4) incubating labeled interacting molecules and the mammalian cells, and selecting labeled mammalian cells by flow cytometry or magnetic-activated sorting;

5) extracting DNAs from the labeled mammalian cells, sequencing the DNAs, and analyzing the change of the sequence ratios before and after sorting;

and, the amino acid mutation in which the ratio after sorting is significantly higher than the ratio before sorting is an amino acid mutation capable of enhancing the binding affinity between the protein and the interacting molecule.

The screening method can efficiently screen various amino acid mutations, has very good convenience and effectiveness, and can be used for screening amino acid mutations which enhance various protein-protein interactions or protein-other molecules interactions.

The beneficial effects of the present disclosure include: the modified Fc region or Fc fragment of the present disclosure has an enhanced affinity to FcγRIIB, and some of them have a reduced affinity to activating FcγRs while having the enhanced affinity to FcγRIIB These proteins can be used to design, modify, or optimize the activation activity of antibodies or fusion proteins. In particular, it is possible to design agonist activity of an antibody or a fusion protein based on a human IgG2 backbone. Thus, it is possible to provide proteins with significantly enhanced agonistic activity, for the treatment of tumors, inflammatory diseases, autoimmune diseases, or a combination thereof, which have a significant application prospect.

### BRIEF DESCRIPTION OF DRAWINGS

FIG. 1 shows the construction of a plasmid for displaying antibodies on the surface of mammalian cells.

FIG. 2 shows a schematic diagram of a mutation screening process. In one embodiment, the process includes: mutation library is constructed and transferred to the cells by retrovirus; the Fc mutant library is expressed on the cell surface; the cells with high affinity to FcγRIIB are sorted; the DNA is extracted and sequenced by high-throughput sequencing (NSG); the enrichment of various mutations after sorting relative to before sorting are analyzed, and the mutations with relatively high enrichment factor compared with the wild type are selected.

FIG. 3 shows the Eu numbering of CH2 amino acids on human IgG2 Fc.

FIG. 4 shows that flow cytometry sorting can gradually enrich cells with high binding affinity to FcγRIIB The cell flow cytometry results of wild-type human IgG2 cells and human IgG2 library LibraryMix cells after one or two rounds of FcγRIIB sorting are shown, respectively.

FIG. 5 shows that the binding affinity of the cells screened from the library to FcγRIIB is significantly improved. The cell flow cytometry results of wild-type human IgG2 cells and cell libraries after two rounds of FcγRIIB sorting are shown, respectively.

FIG. 6 shows a schematic diagram of two types of combined mutation libraries. Type I: combined point mutation among four regions, i.e., regions P233-V240, V266-P271, S298-T299 and G327-I332 each has 0-1 mutation, and the combined mutation contains 0-4 point mutations. Type II: combined mutation of multiple amino acids in one region, i.e., regions P233-V240, V266-P271, S298-T299 or G327-I332 has combined mutation inside each region (respectively 0-7, 0-6, 0-2 and 0-6 combined mutations).

FIG. 7 shows the flow cytometry results of the library IgG2_C01 and the library IgG2_C02 after flow cytometry sorting and the wild-type IgG2. The ratio of the cells binding to FcγRIIB in the library IgG2_C01 and the library IgG2_C02 is improved.

FIG. 8 shows the binding affinity of library Cmix and library C2 cells to FcγRIIB before (“_befor”) and after (“_1^{st}” and “_2^{nd}”) flow cytometry sorting. The results show that the ratio of cells binding to FcγRIIB in the sorted library was gradually increased (2^{nd}>1^{st}>befor).

FIG. 9 shows the binding affinity of different IgG2 antibody Fc mutants to FcγRI (expressed as ELISA signal: absorbance at 650 nm (A650)).

FIG. 10 shows the binding affinity of different IgG2 antibody Fc mutants to FcγRIIA^{131H} (expressed as ELISA signal: absorbance at 650 nm (A650)).

FIG. 11 shows the binding affinity of different IgG2 antibody Fc mutants to FcγRIIA^{131R} (expressed as ELISA signal: absorbance at 650 nm (A650)).

FIG. 12 shows the binding affinity of different IgG2 antibody Fc mutants to FcγRIIB (expressed as ELISA signal: absorbance at 650 nm (A650)).

FIG. 13 shows the binding affinity of different IgG2 antibody Fc mutants to FcγRIIIA^{158F} (expressed as ELISA signal: absorbance at 650 nm (A650)).

FIG. 14 shows the binding affinity of different IgG2 antibody Fc mutants to FcγRIIIA^{158V} (expressed as ELISA signal: absorbance at 650 nm (A650)).

FIG. 15 shows the binding affinity of the IgG2 mutants corresponding to IgG1 V11(hIgG1V11) to FcγRIIA^{131H} and FcγRIIB by ELISA (expressed as ELISA signal: absorbance at 650 nm (A650)).

FIG. 16 shows the immune activation activity analysis results of different antibody Fc mutant anti-human OX40 antibodies, expressed as a column diagram of the mean fluorescence intensity of CD4 positive cell CFSE (the lower the CFSE signal, the higher the activity).

FIG. 17 shows the immune activation activity analysis results of different antibody Fc mutant anti-human OX40 antibodies, expressed as the fluorescence intensity histogram of CD4 positive cell CFSE (the lower the CFSE signal, the higher the activity).

### DETAILED DESCRIPTION

Unless otherwise stated, the scientific and technical terms used herein shall be understood as having the meanings as commonly understood. Unless otherwise required, as used herein, terms in singular forms include the meaning of plurality, and vice versa. In general, as used herein, nomenclatures and techniques in the cell and tissue culturing/incubation, molecular biology, immunology, protein and nucleic acid chemistry described herein are those already known and commonly used in the art.

Methods and techniques used in the present disclosure are generally performed as in conventional practices and as described in general and specialized references cited and discussed in this disclosure, unless otherwise indicated. For example, reference can be made to the followings: Molecular Cloning: A Laboratory Manual, Sambrook et al., the 2^{nd} Edition, Cold Spring Harbor Laboratory Press, Cold Spring Harbor, N.Y., 1989; Current Protocols in Molecular Biology, Ausubel et al. Greene Publishing Associates, 1992; Antibodies: A Laboratory Manual, Harlow and Lane, Cold Spring Harbor Laboratory Press, Cold Spring Harbor, N.Y., 1990, all being incorporated by reference in their entirety. Enzymatic reactions and purifications are performed by following manufacturers' instructions or as known in the art or as specified in the present disclosure. As used herein, nomenclatures, test methods and techniques in biology, pharmacology, medicine and pharmaceutical chemistry are those already known and commonly used in the art. Chemical synthesis, chemical analysis, pharmaceutical manufacturing, formulation and delivery and patient treatment all follow standard practices.

Unless otherwise stated, the terms have the meanings as defined below.

An “antibody” (Ab) should include, but is not limited to, a glycoprotein immunoglobulin or antigen-binding portions thereof. Antibodies In one embodiment bind to corresponding antigens, and each includes at least two heavy (H) chains and two light (L) chains that bind to one another through disulfide bonds. Each heavy chain includes a heavy chain variable region (VH) and a heavy chain constant region. The “heavy chain constant region” or CDs of antibodies include three domains identified as CH1, CH2 and CH3, as well as a hinge region between the CH1 domain and the CH2 domain. Each light chain includes a light chain variable region (VL) and a light chain constant region. The light chain constant region consists of one domain CL. VH and VL can each be subdivided into hypervariable regions called “complementary determining regions (CDRs)” and the interspersed relatively conserved regions called “frame regions (FRs)”. VH and VL are each composed of three CDRs and four FRs as arranged in the following order from the amino end to the carboxyl end: FR1, CDR1, FR2, CDR2, FR3, CDR3, FR4. The heavy chain and the light chain variable regions provide the domains that interact with antigens.

“Fc region” (crystallizable fragment region) or “Fc domain” or “Fc” refers to the C-terminal portion of the heavy chain, which mediates the binding between the immunoglobulin and host tissues or factors, including the binding to Fc receptors on various immune cells (e.g., effector cells) or to the first component of the classic complement system (C1q). Thus, the Fc region is a polypeptide that forms the part of the antibody heavy chain constant region other than the first constant region immunoglobulin domain (CH1 domain). For the allotypes of IgG, IgA and IgD antibodies, the Fc region is composed of two identical protein fragments from CH2 and CH3 domains of the two heavy chains; and in IgM and IgE, the Fc region includes three heavy chain constant domains (CH2-CH3-CH4 domains) in each polypeptide chain. For IgG, the Fc region contains the hinge region between the immunoglobulin domains CH2 and CH3 and the hinge region between CH1 and CH2. Though boundaries of Fc regions of heavy chains of immunoglobulins are variable, Fc regions in heavy chains of human IgGs are normally defined as the segment spanning position P231 to the carboxyl end of the heavy chains, as numbered according to the EU index, the same as in the Kabat system. The CH2 domain of the human IgG Fc region extends from about amino acid 231 to about amino acid 340, while the CH3 domain is located at the C-terminal of the CH2 domain of the Fc region, i.e., it extends from about amino acid 341 of IgG to about amino acid 447.

The “Fc polypeptide fragment” refers to a fragment contained in the Fc region. The Fc polypeptide fragment herein refers to a fragment extending from the amino acid 233 to the amino acid 332 in the Fc region, or other fragments within the Fc region containing said fragment, such as the Fc CH2 domain or the Fc region.

As used herein, “Fc region” or “Fc polypeptide fragment” may be a natural Fc or a modified Fc. In addition, “Fc region” or “Fc polypeptide fragment” may also refer to such area in a separated state, or such area provided in a protein polypeptide comprising Fc, which is also referred to as an “Fc fusion protein” (e.g., an antibody or an immunoadhesin).

“Fc receptor” or “FcR” is the receptor binding to the Fc region of an immunoglobulin. FcRs binding to IgG antibodies include the FcγR family, including their allelic variants and alternative splicing variants. Members of the human Fcγ receptor family include FcγRI (CD64), FcγRIIA (CD32a), FcγRIIB (CD32b), FcγRIIIA (CD16a), FcγRIIIB (CD16b). Therein, FcγRIIB is the only inhibitory Fcγ receptor, and the rest are all activating Fcγ receptors. Most natural effector cells co-express one or more activating FcγRs and the inhibitory FcγRIIB, while natural killer (NK) cells selectively express an activating Fcγ receptor (FcγRIII in mice, FcγRIIIA in humans), while not the inhibitory FcγRIIB in mice and humans. These Fcγ receptors have different molecular structures and therefore different affinity to subclasses of IgG antibodies. Among these Fcγ receptors, FcγRI is a high-affinity receptor, while FcγRIIA, FcγRIIB and FcγRIIIA are low-affinity receptors. Genetic polymorphism is seen in these different Fcγ receptors, which also influences binding affinity. Most dominant genetic polymorphisms include the R131/H131 in FcγRIIA and the V158/F158 in FcγRIIIA Some of these polymorphisms are found disease-associated. And, responsiveness to certain therapeutic antibodies depends on the presence of certain polymorphisms in Fcγ receptors.

As used herein, a “sequence” should be understood as encompassing sequences that are substantially identical to a specified one according to the disclosure. The term “substantially identical sequences” refers sequences sharing a sequence identity of at least 70, 75 or 80%, preferably at least 90 or 95%, more preferably at least 97, 98 or 99% in the optimal alignment using, for example, GAP or BESTFIT and the default gap value. In one embodiment, the differential residues are conservative amino acid substitution. The term “conservative amino acid substitution” refers to substitution between amino acid residues having similar chemistry (e.g., charge or hydrophilicity) in the side-chain R group. Generally, conservative amino acid substitution does not substantially change a protein's functional properties. When difference(s) between two or more amino acid sequences reside(s) in conservative substitution(s), the sequence identity percentage or similarity value can be up-adjusted to correct for the conservativeness of substitution. See, for example, Pearson, Methods Mol. Biol. 243: 307-31(1994). Examples of amino acids having side-chains with similar chemistry include: 1) aliphatic side chains, including glycine, alanine, valine, leucine and isoleucine; 2) aliphatic-hydroxy side chains, including serine and threonine; 3) amide-containing side chains, including asparagine and glutamine; 4) aromatic side chains, including phenylalanine, tyrosine and tryptophan; 5) basic side chains, including lysine, arginine and histidine; 6) acidic side chains, including aspartic acid and glutamic acid; and 7) sulfur-containing side chain, including cysteine and methionine. conservative amino acid substitutions include valine-leucine-isoleucine, phenylalanine-tyrosine, lysine-arginine, alanine-valine, glutamate-aspartate, and asparagine-glutamine. As noted above, in addition to the amino acid mutations provided by the present disclosure, the Fc region, the antibody, the Fc fragment or the fusion protein according to the present disclosure may further include other possible modifications not disclosed in the present disclosure as long as the “substantially identical” requirements described above are met.

Amino acids of the antibody and a fragment or domain thereof according to the disclosure are numbered according to the Eu numbering system for IgGs. The mutant types of some mutants involved in the present disclosure and their nomenclature are shown in Table 1.

Antibodies usually In one embodiment bind to the corresponding antigens with a high affinity (likewise, the proteins of the disclosure may also In one embodiment bind to their epitope recognition molecules), as indicated by, for example, a dissociation constant (KD) of 10⁻⁵-10⁻¹¹ M or less. A KD above about 10⁻⁴ M⁻¹ is usually understood as indicating a nonspecific binding. In this disclosure, an antibody “In one embodiment binding to” an antigen means that the antibody binds to the antigen or a substantially identical antigen with a high affinity as measured by a KD of 10⁻⁷M or less, preferably 10⁻⁸M or less, more preferably 5×10⁻⁹M or less and most preferably 10⁻⁸-10⁻¹° M or less, and does not bind to any irrelevant antigen with a significant affinity. An antigen is “substantially identical” to a specified antigen, if they share a high sequence identity, such as a sequence identity of at least 80%, at least 90%, preferably at least 95%, more preferably at least 97%, or even more preferably at least 99%.

Immunoglobulins may come from any commonly known isoform. IgG isoforms can be subdivided into subclasses in some species: IgG1, IgG2, IgG3 and IgG4 in human, IgG1, IgG2a, IgG2b, and IgG3 in mouse. The term “isotype” refers to antibodies encoded by heavy chain constant region genes (e.g. IgM or IgG1). The “antibodies” include naturally occurring and non-naturally occurring antibodies, monoclonal and polyclonal antibodies, chimeric and humanized antibodies, human and non-human antibodies, wholly synthetic antibodies, and single chain antibodies.

The “natural IgG” described in the present disclosure refers to those IgG that can be naturally present, the sequences of which are not artificially modified. It should be noted that, due to the polymorphism of the gene, even if the natural IgG may also have different mutants. Any non-artificially modified IgG in the natural state is referred to as natural IgG, whether there is a mutation or not.

An “agonistic antibody” is an antibody that binds to and activates a receptor. Examples of such agonism include an agonistic antibody that binds to a receptor of a tumor necrosis factor receptor (TNFR) superfamily and induces cell apoptosis expressing a TNF receptor. Assays for determining apoptosis-inducing effects are described in WO98/51793 and WO99/37684, which are particularly incorporated herein by reference. In a specific embodiment of the present disclosure, the anti-CD40 agonistic antibody is capable of enhancing the anti-tumor immune response to indirectly kill tumor cells by binding to the target molecule transmitting the immune activation signal on the immune cell surface and activating the important immune activation signal pathway controlled by the target molecule. Examples of agonistic antibodies that have entered the clinical study stage may be referred to in PCT/CN2017/087620.

The term “agonistic activity” refers to the activity of inducing certain specific physiological activity changes by binding an antibody to an antigen molecule and exciting the antigen molecule to generate a signal. Antigen molecules include receptor molecules and other molecules with signal or physiological functions. Said specific physiological activities include but are not limited to: proliferation activity, survival activity, differentiation activity, transcriptional activity, membrane transport activity, affinity, protein decomposition activity, phosphorylation/dephosphorization activity, redox activity, transfer activity, nucleic acid decomposition activity, dehydration activity, cell death induction activity, apoptosis induction activity, and the like.

The term “parent” refers to an object that is modified during protein modification. The parent may be a naturally occurring polypeptide, or a variant or modified version of a naturally occurring polypeptide. In some embodiments, the parental Fc region of the present disclosure is an Fc region of natural IgG.

A “variant” refers to a protein obtained after modification of a parental protein in a protein modification process. In one embodiment, variants can be proteins that are derived from the parental protein by mutations, deletions and/or additions of amino acids in the parental protein, and retain some or all of the function of the parent. In one embodiment, sequence of a mutant herein has at least about 80% homology to the parent's sequence, most preferably at least about 90% homology, more preferably at least about 95% homology. Accordingly, the term “Fc variant” as used herein refers to an Fc sequence that differs from the parent Fc sequence due to at least one amino acid modification. The Fc variant may include only the Fc region, or may be present in the context of an antibody, Fc fusion, isolated Fc, Fc region, or other polypeptide that is substantially encoded by Fc. The Fc variant may refer to the Fc polypeptide itself, a composition comprising an Fc variant polypeptide, or an amino acid sequence encoding it.

As used herein, “tumor necrosis factor receptor superfamily” and “TNF receptor superfamily” both refer to receptor polypeptides that bind to cytokines of the TNF family. Generally, these receptors are type I transmembrane receptors comprising one or more cysteine-rich repeats in their extracellular domain. Examples of cytokines of TNF family include tumor necrosis factor-α (TNF-α), tumor necrosis factor-β (TNF-β or lymphotoxin), CD30a ligand, CD27a ligand, CD40a ligand, OX-40a ligand, 4-1BB ligand, Apo-1 ligand (also known as Fas ligand or CD95 ligand), Apo-2 ligand (also known as TRAIL), Apo-3 ligand (also known as TWEAK), osteoprotegerin (OPG), APRIL, RANK ligand (also known as TRANCE) and TALL-1 (also known as BlyS, BAFF or THANK). Examples of receptors of the TNF receptor superfamily include type 1 tumor necrosis factor receptor (TNFR1), type 2 tumor necrosis factor receptor (TNFR2), p75 nerve growth factor receptor (NGFR), B-cell surface antigen CD40, T-cell antigen OX-40, Apo-1 receptor (also known as Fas or CD95), Apo-3 receptor (also known as DR3, sw1-1, TRAMP and LARD), receptors called “transmembrane activator and CAML-interactor” or “TACI”, BCMA protein, DR4, DR5 (also known as Apo-2; TRAIL-R2, TR6, Tango-63, hAPO8, TRICK2 or KILLER), DR6, DcR1 (also known as TRID, LIT or TRAIL-R3), DcR2 (also known as TRAIL-R4 or TRUNDD), OPG, DcR3 (also known as TR6 or M68), CAR1, HVEM (also known as ATAR or TR2), GITR, ZTNFR-5, NTR-1, TNFL1, CD30, lymphotoxin β receptor (LTBr), 4-1BB receptor and TR9 (EP988,371 A1).

As used herein, the terms “ratio of the affinity to the inhibitory Fcγ receptor to the affinity to the activating Fcγ receptor” and “I/A ratio” both refer to the ratio of the antibody heavy chain constant region's affinity to the inhibitory Fcγ receptor (such as human FcγRIIB) to its highest affinity to the activating Fcγ receptor of the same species, e.g., human FcγRI, FcγRIIA, FcγRIIIA, FcγRIIIB

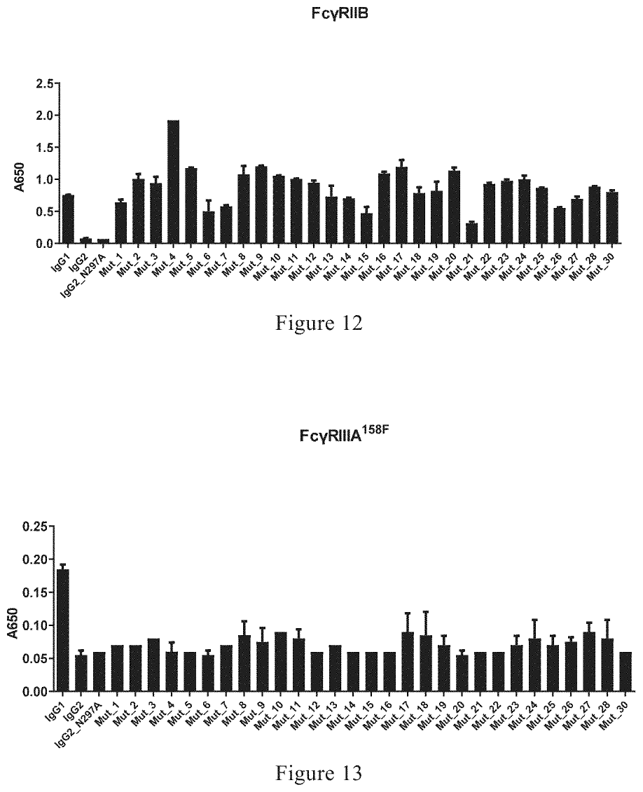

The term “affinity” refers to the capability of two molecules binding to each other, as usually measured by a KD value. Affinity herein can also be measured by an enrichment factor (as shown in Table 7) or binding analysis signal (as shown in FIG. 12). The term “KD” refers to the equilibrium dissociation constant of interaction between two molecules (e.g., a specific antibody vs. an antigen or a specific ligand vs. a receptor). The term “enrichment factor” refers to the ratio of the percentage of the cells expressing the antibody containing the specific Fc mutation after sorting to such percentage before sorting (analyzed by second-generation sequencing, as shown in Table 7), and the sorting is flow cytometry sorting or magnetic-activated sorting provided by the present disclosure. The “binding analysis signal” is the absorbance value in the ELISA analysis provided by the present disclosure (as shown in FIG. 12).

“Human” antibodies refer to such antibodies and the variable regions include FRs and CDRs from human immunoglobulins. And, the antibody may also include a constant region from a human immunoglobulin, if present. A human antibody according to the disclosure may include amino acid residues not encoded by human immunoglobulin sequences, such as mutations introduced by random or site-directed mutagenesis in vitro or somatic mutation in vivo. However, as used herein, the term “human antibody” has no intention to include such antibodies that include non-human mammalian (e.g., mouse) CDR sequences grafted between human frame sequences. The term “human” antibody and “wholly human” antibody are used as synonymous to each other.

The “antibodies” of the disclosure include naturally occurring and non-naturally occurring antibodies, monoclonal and polyclonal antibodies, chimeric and humanized antibodies, human and non-human antibodies and wholly synthetic antibodies.

A “humanized” antibody refers to an antibody and some, majority or all of the amino acids outside the non-human CDR domains are replaced by corresponding amino acids from a human immunoglobulin. In an embodiment of a humanized antibody, some, majority or all of the amino acids outside the CDR domains are replaced by corresponding amino acids from a human immunoglobulin, and some, majority or all of the amino acids in one or more of the CDRs remain unchanged. Minor addition, deletion, insertion, substitution or modification of amino acid(s) is tolerable, as long as it does not destroy the antibody's capability of binding to the corresponding antigen. A “humanized” antibody retains the antigen binding specificity of the original antibody.

A “chimeric antibody” refers to an antibody and the variable region(s) come(s) from a species and the constant region(s) from another, for example, an antibody in which the variable region(s) may come from a mouse antibody and the constant region(s) from a human antibody.

The disclosure is further illustrated by the specific examples described below. It should be understood that these examples are merely illustrative, and do not limit the scope of the present disclosure. The experimental methods without specifying the specific conditions in the following examples generally used the conventional conditions, such as those described in Sambrook, Molecular Cloning: A Laboratory Manual (New York: Cold Spring Harbor Laboratory Press, 1989), or followed the manufacturer's recommendation. Unless otherwise stated, the percentages and parts are by weight.

TABLE 1
Mutation types of some mutants involved in the
present disclosure and their nomenclature

| Name | IgG2 mutation |
|---|---|
| Mut_1 | L328W |
| Mut_2 | H268D/S298L/L328W |
| Mut_3 | S267V/S298L/L328W |
| Mut_4 | V234M/S267E/S298L/L328W |
| Mut_5 | A235W/V266L/S298L/L328W |
| Mut_6 | V234Q/A235G/P238L/S239V/H268D/G327A/L328E/A330S/ |
| | I332T |
| Mut_7 | S239V/V266L/S298L/L328W |
| Mut_8 | V266L/L328W |
| Mut_9 | V266L/S267D/H268D/E269D/P271Q |
| Mut_10 | S267E/H268S/E269D |
| Mut_11 | S267E/A330S |
| Mut_12 | P233F/V266L/S298L/L328W |
| Mut_13 | V266L/S298L/L328W |
| Mut_14 | V234M/S267E/S298G/I332L |
| Mut_15 | V234Q/A235G/P238L/S239V/G327A/L328E/A330S/I332T |
| Mut_16 | S267E/S298G |
| Mut_17 | V266L/S267E/H268S/E269V |
| Mut_18 | P233G/S267E |
| Mut_19 | V266L/S267E/H268S/E269V/P271C |
| Mut_20 | V266L/S267E/H268S/E269A/P271C |
| Mut_21 | S267V/S298G/L328W |
| Mut_22 | V266L/S267M/H268E/P271Q |
| Mut_23 | V266L/S267E/E269K/P271G |
| Mut_24 | V266L/S267E/H268S/E269G/P271T |
| Mut_25 | V234Q/V266L/S267D/H268D/E269D/P271Q |
| Mut_26 | V234Q/A235G/P238L/S239V/S267E/H268S/E269D |
| Mut_27 | V266L/S267E/H268S/P271V |
| Mut_28 | P233G/S267E/S298G |
| Mut_29 | V234Q/A235G/P238L/S239V/S267E/S298L/G327A/L328E/ |
| | A330S/I332T |
| Mut_30 | V234Q/V266L/S267E/H268S/E269V |

Materials and Methods

Construction of Vector for Mammalian Cell Surface Display

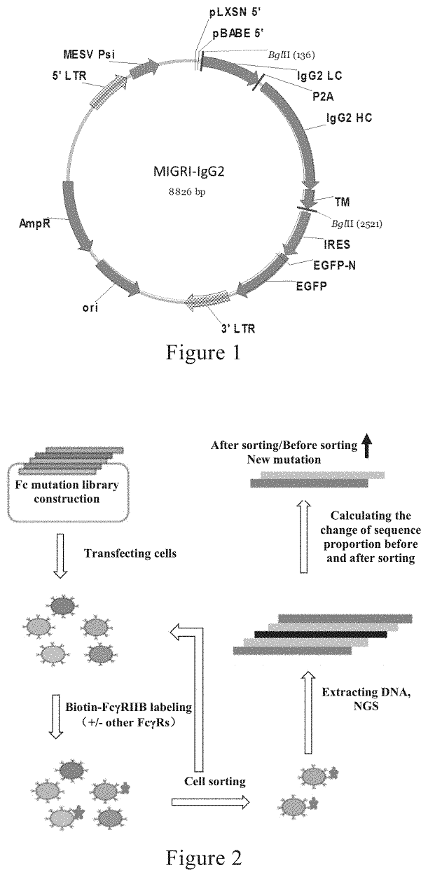

The light and heavy-chain full-length sequences of human IgG2 anti-mouse CD40 antibody are constructed to retrovirus vector MIGRI (Addgene, Pear et al. Blood. 1998 Nov. 15. 92(10):3780-92) by Multi One Step Cloning Kit (Yeasen Biotechnology (Shanghai) Co., Ltd., China). The primers used are shown in Table 2, and the construction is shown in FIG. 1. The vector is digested by restriction endonuclease BglII, into which light chain DNA and heavy chain DNA (connected by a self-cleaving Linker P2A) of the complete antibody are inserted; The end of the heavy chain DNA includes a transmembrane domain DNA (DNA sequence of each fragment is shown in Table 3). The plasmid expresses the GFP protein while expressing the antibody human IgG2. The amino acid sequences of the surface display antibody and the related Fc region thereof are as shown in Table 4. The construct is verified by sequencing.

TABLE 2
Primers used in construction of antibody-
expressing vectors for mammalian cell
surface display

| | | |
|---|---|---|
| SEQ ID NO: 1 | 5MIGR1- | CTAGGCGCCGGAATTAGATCTACCA |
| | Bg1II-LC | TGGAGACAGACAGAC |
| SEQ ID NO: 2 | 3′LC-P2 | GTTTGTTGCGCCGGATCCACACTCT |
| | | CCCCTGTTGAA |
| SEQ ID NO: 3 | 5′LC-P2 | TTCAACAGGGGAGAGTGTGGATCCG |
| | | GCGCAACAAAC |
| SEQ ID NO: 4 | 3′P2-HC | GCTGAGCCTGATGTCCATGGGTCCA |
| | | GGATTCTCTTC |
| SEQ ID NO: 5 | 5′P2-HC | GAAGAGAATCCTGGACCCATGGACA |
| | | TCAGGCTCAGC |
| SEQ ID NO: 6 | 3′HC-TM | GGATGGAGGAGGCTCGAGTTTACCC |
| | | GGAGACAGGGA |
| SEQ ID NO: 7 | 5′HC-TM | TCCCTGTCTCCGGGTAAACTCGAGC |
| | | CTCCTCCATCC |
| SEQ ID NO: 8 | 3MIGR1- | TTCGTTAACCTCGAGAGATCTTCAC |
| | Bg1II-TM | GCTAGAGAATGAGG |

TABLE 3
DNA sequence of each fragment of the
surface displayed antibodies

Antibody light chain DNA sequence (SEQ ID
NO: 9)
ATGGAGACAGACAGACTCCTGCTATGGGTGCTGCTGCTCTGGGTGCCAGG
CTCCACTGGTGACACTGTACTGACCCAGTCTCCTGCTTTGGCTGTGTCTC
CAGGAGAGAGGGTTACCATCTCCTGTAGGGCCAGTGACAGTGTCAGTACA
CTTATGCACTGGTACCAACAGAAACCAGGACAGCAACCCAAACTCCTCAT
CTATCTAGCATCACACCTAGAATCTGGGGTCCCTGCCAGGTTCAGTGGCA
GTGGGTCTGGGACAGACTTCACCCTCACCATTGATCCTGTGGAGGCTGAT
GACACTGCAACCTATTACTGTCAGCAGAGTTGGAATGATCCGTGGACGTT
CGGTGGAGGCACCAAGCTGGAATTGAAACGAACTGTGGCTGCACCATCTG
TCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAG
AGCAGGACAGCAAGGACAGCACCTACAGCCTCAGCAGCACCCTGACGCTG
AGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
Self-cleaving Linker P2A DNA sequence (SEQ
ID NO: 10)
GGATCCGGCGCAACAAACTTCTCTCTGCTGAAACAAGCCGGAGATGTCGA
AGAGAATCCTGGACCC
Antibody heavy chain DNA sequence (SEQ ID
NO: 11)
ATGGACATCAGGCTCAGCTTGGTTTTCCTTGTCCTTTTCATAAAAGGTGT
CCAGTGTGAGGTGCAGCTGGTGGAGTCTGATGGAGGCTTAGTGCAGCCTG
GAAGGTCCCTAAAACTCCCCTGTGCAGCCTCAGGATTCACTTTCAGTGAC
TATTACATGGCCTGGGTCCGCCAGGCTCCAACGAAGGGGCTGGAGTGGGT
CGCAAGCATTAGTTATGATGGTAGTAGCACTTACTATCGAGACTCCGTGA
AGGGCCGATTCACTATCTCCAGAGATAATGCAAAAAGCACCCTATACCTG
CAAATGGACAGTCTGAGGTCTGAGGACACGGCCACTTATTACTGCGGAAG
ACACAGTAGCTACTTTGATTACTGGGGCCAAGGAGTCATGGTCACAGTCT
CGAGCGCCTCCACCAAGGGCCCATCGGTCTTCCCCCTGGCGCCCTGCTCC
AGGAGCACCTCCGAGAGCACAGCGGCCCTGGGCTGCCTGGTCAAGGACTA
CTTCCCCGAACCGGTGACGGTGTCGTGGAACTCAGGCGCTCTGACCAGCG
GCGTGCACACCTTCCCAGCTGTCCTACAGTCCTCAGGACTCTACTCCCTC
AGCAGCGTGGTGACCGTGCCCTCCAGCAACTTCGGCACCCAGACCTACAC
CTGCAACGTAGATCACAAGCCCAGCAACACCAAGGTGGACAAGACAGTTG
AGCGCAAATGTTGTGTCGAGTGCCCACCGTGCCCAGCACCACCTGTGGCA
GGACCGTCAGTCTTCCTCTTCCCCCCAAAACCCAAGGACACCCTCATGAT
CTCCCGGACCCCTGAGGTCACGTGCGTGGTGGTGGACGTGAGCCACGAAG
ACCCCGAGGTCCAGTTCAACTGGTACGTGGACGGCGTGGAGGTGCATAAT
GCCAAGACAAAGCCACGGGAGGAGCAGTTCAACAGCACGTTCCGTGTGGT
CAGCGTCCTCACCGTTGTGCACCAGGACTGGCTGAACGGCAAGGAGTACA
AGTGCAAGGTCTCCAACAAAGGCCTCCCAGCCCCCATCGAGAAAACCATC
TCCAAAACCAAAGGGCAGCCCCGAGAACCACAGGTGTACACCCTGCCCCC
ATCCCGGGAGGAGATGACCAAGAACCAGGTCAGCCTGACCTGCCTGGTCA
AAGGCTTCTACCCCAGCGACATCGCCGTGGAGTGGGAGAGCAATGGGCAG
CCGGAGAACAACTACAAGACCACACCTCCCATGCTGGACTCCGACGGCTC
CTTCTTCCTCTACAGCAAGCTCACCGTGGACAAGAGCAGGTGGCAGCAGG
GGAACGTCTTCTCATGCTCCGTGATGCATGAGGCTCTGCACAACCACTAC
ACGCAGAAGAGCCTCTCCCTGTCTCCGGGTAAA
Transmembrane domain DNA sequence (SEQ ID
NO: 12)
CTCGAGCCTCCTCCATCCACTGTCTCCAACATGGCGACCGTTGCTGTTCT
GGTTGTCCTTGGAGCTGCAATAGTCACTGGAGCTGTGGTGGCTTTTGTGA
TGAAGATGAGAAGGAGAAACACAGGTGGAAAAGGAGGGGACTATGCTCTG
GCTCCAGGCTCCCAGACCTCTGATCTGTCTCTCCCAGATTGTAAAGTGAT
GGTTCATGACCCTCATTCTCTAGCGTGA

TABLE 4
Amino acid sequence of the surface display
antibody and the related Fc region thereof

Amino acid sequence of the human IgG2
anti-mouse CD40 surface displayed
antibody (SEQ ID NO: 13):
METDRLLLWVLLLWVPGSTGDTVLTQSPALAVSPGERVTISCRASDSVST
LMHWYQQKPGQQPKLLIYLASHLESGVPARFSGSGSGTDFTLTIDPVEAD
DTATYYCQQSWNDPWTFGGGTKLELKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGECGSGATNFSLLKQAGDVE
ENPGPMDIRLSLVFLVLFIKGVQCEVQLVESDGGLVQPGRSLKLPCAASG
FTFSDYYMAWVRQAPTKGLEWVASISYDGSSTYYRDSVKGRFTISRDNAK
STLYLQMDSLRSEDTATYYCGRHSSYFDYWGQGVMVTVSSASTKGPSVFP
LAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDIAVEW
ESNGQPENNYKTTPPMLDSDGSFFLYSKLTVDKSRWQQGNVFSCSVMHEA
LHNHYTQKSLSLSPGKLEPPPSTVSNMATVAVLVVLGAAIVTGAVVAFVM
KMRRRNTGGKGGDYALAPGSQTSDLSLPDCKVMVHDPHSLA
Amino acid sequence of the Fc region of
the human IgG2 anti-mouse CD40 surface
displayed antibody (SEQ ID NO: 14):
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDIAVEW
ESNGQPENNYKTTPPMLDSDGSFFLYSKLTVDKSRWQQGNVFSCSVMHEA
LHNHYTQKSLSLSPGK
Amino acid sequence of the Fc CH2
domain of the human IgG2 anti-mouse
CD40 surface displayed antibody (SEQ
ID NO: 15):
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTK
Amino acid sequence corresponding to the
residues 233rd to 332nd of Fc region of
IgG2 antibody according to Eu numbering
(SEQ ID No: 20):
PVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPI

Construction of Fc-Mutant Library

Point mutation libraries are designed to introduce mutations through degenerate primers, and multiple degenerate primers are designed. For example, the degenerate primer used for the P233 point mutation is CCACCGTGCCCAGCACCANNSGTGGCAGGACCGTCAGTC (SEQ ID NO: 21). The mutant library fragment and the connection vector are obtained by PCR, and ligated by Multi One Step Cloning Kit (Yeasen Biotechnology (Shanghai) Co., Ltd., China) and transformed into DH5a to obtain a mutant library. For each library, 20 colonies were picked for sequencing to confirm whether a mutation was introduced at the target location.

The method for the combined mutation library is similar. The primers are designed according to the mutation type with enrichment which has been screened according to the point mutation library. The intermediate degenerate primer part is modified to be a primer sequence comprising a codon encoding a specific amino acid or a degenerate codon encoding several amino acids, for example, L328W (TGCAAGGTCTCCAACAAAGGCTGGCCAGCCCCCATCGAGAAAAC, SEQ ID NO: 22), Combine2_1 (ACGTGCGTGGTGGTGGACKNGWBGVASRWSSACCHGGAGGTCCAGTTC AACTGG, SEQ ID NO: 23).

In addition to the normal base, R in the above primers represents A/G, Y represents C/T, M represents A/C, K represents G/T, S represents G/C, W represents A/T, H represents A/T/C, B represents G/T/C, V represents G/A/C, D represents G/A/T, and N represents A/T/C/G.

Construction of Stably-Expressing Cell Lines

The library plasmid and the helper plasmid Phoenix-Eco (molar ratio 2:1) were mixed with transfection reagent PEI (mass ratio 1:5) and added into plated Phoenix cells (confluence 80%), changing fresh culture after 6 hours, and the supernatant containing retrovirus was collected after 48 hours. Plated 3T3 cells with a confluence of 80% prepared in advance were infected by 50% fresh medium plus 50% of the above retrovirus supernatant. The solution was changed after 24 hours. After 48 hours, the cell line stably expressing the antibody can be obtained.

Flow Cytometry Analysis of the Cell Lines Stably Expressing the Antibody

3T3 cells were collected by trypsin digestion, and a single cell suspension was prepared. About 1-5*10⁶ cells were re-suspended in 50 μl FACS buffer (1×PBS containing 0.5% FBS and 2 mM EDTA) supplied with PE-F(ab′)2 Fragment Goat Anti-Human IgG (1:500, Jackson ImmunoResearch Laboratories) or Biotin-FcγRIIB (1 μg/ml, Sino Biological) and Streptavidin-APC (1:500, BD Biosciences). The cells were incubated for 15 minutes on ice and then washed twice with FACS buffer and re-suspended in 200 μl FACS buffer and analyzed by flow cytometry.

Sorting of Cells with High Affinity to FcγRIIB by Flow Cytometry

The staining system is amplified according to the above-mentioned flow cytometry analysis and staining method, and the cell is re-suspended to the final concentration of ˜1-5*10⁷ cells/ml before loading. Cells expressing IgG (strong PE fluorescence signal) and having high affinity to FcγRIIB (strong APC fluorescence signal) are sorted, and the proportion is controlled to be less than 1% of IgG positive cells.

Magnetic-Activated Sorting of Cells with High Affinity to FcγRIIB and Low Affinity to Activating FcγR (High I/A Ratio)

3T3 cells were collected by trypsin digestion, and a single cell suspension was prepared. About 1*10⁷ cells were re-suspended in 50 μl MACS buffer (1×PBS containing 0.5% BSA and 2 mM EDTA) supplied with Biotin-FcγRIIB (1 μg/ml, Sino Biological) and activating FcγRs (including FcγRI, FcγRIIA^{131H}, FcγRIIA^{131R}, FcγRIIIA^{158V}, FcγRIIIA¹⁵⁸F, all for 1 μg/ml, Shanghai NOVOPROTEIN SCIENTIFIC INC.). The cells were incubated for 15 minutes on ice and then washed twice with MACS buffer and re-suspended in 100 μl MACS buffer. Twenty-five μl anti-Biotin magnetic beads were added, and the cells were incubated for 15 minutes on ice and then washed once with MACS buffer and re-suspended to 500 μl, and added to the LS column put in the magnetic field. The effluent was collected, washed with 3 mL of MACS buffer and collected; The LS column was then removed out of the magnetic field, and 5 mL of MACS buffer was used to rapidly elute the remaining cells from the sorting column to collect positive cells, which are the high I/A ratio cells.

High Throughput Sequencing Analysis

About 0.1-5*10⁶ cells of the target cell population were taken and subject to cell lysis genomic DNA extraction, PCR (amplification of Fc fragment). Library construction and high-throughput sequencing (PE 150 or PE 250) were performed, and about 60,000-300,000 sequences were obtained for each sample. The nucleotide sequences of the mutation region were extracted and translated into the corresponding amino acid sequences (http://www.cbs.dtu.dk/services/VirtualRibosome/, Rasmus Wernersson. Nucl. Acids Res. 2006 34: W385-W388). The proportion of each mutation sequence in each sample was calculated, and: the proportion of the mutation sequence=the mutation sequence count/the total sequence count.

Antibody Expression and Purification

An advantageous Fc mutant or a new combined mutant according to NGS is constructed into the heavy chain of the anti-human OX40 antibody, which is transiently transfected into HEK293S cells together with the corresponding light chain to express proteins. Protein G purification is performed to obtain different Fc mutant antibodies, and SDS-PAGE is used to confirm that all the antibody structures are intact. The anti-human OX40 antibody DNA sequence and protein sequence are shown in Table 5 and Table 6.

TABLE 5
DNA sequence of wild-type IgG2 anti-human
OX40 antibody

Antibody light chain DNA sequence (SEQ ID
NO: 16)
ATGGAAACCCCAGCGCAGCTTCTCTTCCTCCTGCTACTCTGGCTCCCAGA
TACCACCGGAGAAATTGTGTTGACGCAGTCTCCAGGCACCCTGTCTTTGT
CTCCAGGGGAAAGAGCCACCCTCTCCTGCAGGGCCAGTCAGAGTGTTAGC
GGCGACTACTTAGCCTGGTACCAGCAGAAACTTGGCCAGGCTCCCAGGCT
CCTCATCTATGGTGCATCCATCAGGGCCACTGGCATCCCAGACAGGTTCA
GTGGCAGTGGGTCTGGGACAGACTTCACTCTCACCATCAGCAAACTGGAG
CCTGAAGATTTTGCAGTGTATTACTGTCAGCAGTATGGTAGCTCACCGCT
CACTTTCGGCGGAGGGACCAAGGTGGAGATCAAACGAACTGTGGCTGCAC
CATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTG
TCACAGAGCAGGACAGCAAGGACAGCACCTACAGCCTCAGCAGCACCCTG
ACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAG
AGTGTTGA
Wild-type IgG2 antibody heavy chain
DNA sequence (SEQ ID NO: 17)
ATGAAACACCTGTGGTTCTTCCTCCTCCTGGTGGCAGCTCCCAGATGGGT
CCTGTCTCAGGTGCAGCTGCAGGAGTCGGGCCCAGGACTGGTGAAGCCTT
CGGGGACCCTGTCACTCACCTGCGCTGTCTCTGGTGGCTCCATCAGCAGT
AGTATCTGGTGGAGCTGGGTCCGCCAGCCCCCAGGGAAGGGGCTGGAATG
GATTGGGGAAATCCATCATAGTGGGAACACCAACTGCAATCCGTCCCTCA
AGAGTCGAGTCACCATATCAATAGACAAGTCCAAGAACCAGTTCTCCCTG
AAACTGACCTCTGTGACCGCCGCGGACACGGCCGTATATTATTGTGCGAG
AGCGGGAACTGGAACTACGCTTGACTACTGGGGCCAGGGAACCCTGGTCA
CCGTCTCGAGCGCCTCCACCAAGGGCCCATCGGTCTTCCCCCTGGCGCCC
TGCTCCAGGAGCACCTCCGAGAGCACAGCGGCCCTGGGCTGCCTGGTCAA
GGACTACTTCCCCGAACCGGTGACGGTGTCGTGGAACTCAGGCGCTCTGA
CCAGCGGCGTGCACACCTTCCCAGCTGTCCTACAGTCCTCAGGACTCTAC
TCCCTCAGCAGCGTGGTGACCGTGCCCTCCAGCAACTTCGGCACCCAGAC
CTACACCTGCAACGTAGATCACAAGCCCAGCAACACCAAGGTGGACAAGA
CAGTTGAGCGCAAATGTTGTGTCGAGTGCCCACCGTGCCCAGCACCACCT
GTGGCAGGACCGTCAGTCTTCCTCTTCCCCCCAAAACCCAAGGACACCCT
CATGATCTCCCGGACCCCTGAGGTCACGTGCGTGGTGGTGGACGTGAGCC
ACGAAGACCCCGAGGTCCAGTTCAACTGGTACGTGGACGGCGTGGAGGTG
CATAATGCCAAGACAAAGCCACGGGAGGAGCAGTTCAACAGCACGTTCCG
TGTGGTCAGCGTCCTCACCGTTGTGCACCAGGACTGGCTGAACGGCAAGG
AGTACAAGTGCAAGGTCTCCAACAAAGGCCTCCCAGCCCCCATCGAGAAA
ACCATCTCCAAAACCAAAGGGCAGCCCCGAGAACCACAGGTGTACACCCT
GCCCCCATCCCGGGAGGAGATGACCAAGAACCAGGTCAGCCTGACCTGCC
TGGTCAAAGGCTTCTACCCCAGCGACATCGCCGTGGAGTGGGAGAGCAAT
GGGCAGCCGGAGAACAACTACAAGACCACACCTCCCATGCTGGACTCCGA
CGGCTCCTTCTTCCTCTACAGCAAGCTCACCGTGGACAAGAGCAGGTGGC
AGCAGGGGAACGTCTTCTCATGCTCCGTGATGCATGAGGCTCTGCACAAC
CACTACACGCAGAAGAGCCTCTCCCTGTCTCCGGGTAAATGA

TABLE 6
Protein sequence of wild-type IgG2 anti-human
OX40 antibody

Antibody light chain protein sequence (SEQ
ID NO: 18)
METPAQLLFLLLLWLPDTTGEIVLTQSPGTLSLSPGERATLSCRASQSVS
GDYLAWYQQKLGQAPRLLIYGASIRATGIPDRFSGSGSGTDFTLTISKLE
PEDFAVYYCQQYGSSPLTFGGGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC
Wild-type IgG2 antibody heavy chain protein
sequence (SEQ ID NO: 19)
MKHLWFFLLLVAAPRWVLSQVQLQESGPGLVKPSGTLSLTCAVSGGSISS
SIWWSWVRQPPGKGLEWIGEIHHSGNTNCNPSLKSRVTISIDKSKNQFSL
KLTSVTAADTAVYYCARAGTGTTLDYWGQGTLVTVSSASTKGPSVFPLAP
CSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEV
HNAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWESN
GQPENNYKTTPPMLDSDGSFFLYSKLTVDKSRWQQGNVFSCSVMHEALHN
HYTQKSLSLSPGK

The Analysis of the Binding Properties of the Mutant Antibody to FcγR by Enzyme-Linked Immunosorbent Assay (ELISA)

One hundred μl (2 μg/mL) of antibody or mutant thereof were added to the enzyme-linked plate to be coated overnight, then the supernatant was discarded. The plate was blocked with PBS containing 1% BSA for 2 hours, and washed with PBST (PBS containing 0.05% Tween 20). Biotin-labeled FcγR extracellular domain protein (Beijing Sino Biological Co., Ltd) of appropriate concentration was added and incubated at room temperature for 1 hour, then the supernatant was discarded and the plate was washed with PBST. The Biotin-labeled protein was detected by incubating with Streptavidin-HRP (BD BioSciences) at room temperature for 1 hour. The supernatant was removed, and the color-developing solution was added for color development for 20-40 minutes to determine the absorbance of 650 nm (A650).

Immune Activation Activity Analysis (Promoting T Cell Proliferation) of Anti-Human OX40 Antibody with Different Antibody Fc Mutant In Vitro

Spleen was isolated from humanized FcγR/OX40 mouse to prepare a single cell suspension after lysing red blood cells. CFSE labeling was performed. Cells were re-suspended in PBS containing 5 μM CFSE to a concentration of 5*10⁶/mL and incubated at 37° C. for 15 minutes. Cells were then washed twice with 5% FBS-containing PBS and spun down at 600 g for 5 min, followed by re-suspending the cells with primary cell culture medium (RPMI+10% FBS+1% Pen/Strep+1% HEPES+1% Sodium Pyruvate+0.1% 2ME (final 50 μM)+1% L-glutamine+1% Non-essential A.A.) to a concentration of 3*10⁶/ml. Each well was added with 100 μl of cell suspension, i.e., 3*10⁵ cells per well. The (anti-hOX40) antibody concentrations were diluted to 2, 0.2 and 0.02 μg/ml, respectively, using the primary cell culture medium containing 0.2 μg/ml anti-CD3. One hundred μl of the prepared antibody mixture was transferred into the wells containing CFSE-labeled humanized FCγR/OX40 spleen cells. Two groups of controls were: CD3 only groups containing CFSE-labeled cells and anti-mouse CD3, but without any other antibodies; and CD3+CD28 groups containing CFSE-labeled cells and anti-mouse CD3 and 2 μg/mL of anti-mouse CD28 antibody (Clone 37.51 (RUO), BD Biosciences) (the final concentrations of anti-mouse CD3 and CD28 were 0.1 μg/ml and 1 μg/ml, respectively). The cells were incubated at 37° C. for 3 days in a 5% CO₂ thermostatic cell incubator. After the culturing was finished, the cells were collected and analyzed for the number and proliferation of CD4⁺ and CD8⁺ cells by flow cytometry. Proliferation of T cells analyzed by flow cytometry: the cultured cells were transferred to a 96-well U bottom plate, washed twice with PBS, centrifuged at 500 g for 5 minutes, then the supernatant was discarded. The cells were re-suspended in 50 μl of FACS buffer (PBS containing 0.5% FBS, 2 mM EDTA) containing PE anti-Mouse CD4 (Clone: GK1.5, 1:500, BD) and APC anti-Mouse CD8a (Clone: 53-6.7, 1:500, BioLegend), incubated on ice in dark for 15 minutes, then washed twice with PBS buffer and re-suspended in 200 μl of FACS buffer containing DAPI (0.5 μg/ml, Invitrogen) and CountBright Absolute Counting Beads (Life Technologies, 2 μl/sample), and analyzed by flow cytometry.

The present disclosure mainly provides antibody Fc mutant sequences that enhance human IgG2 interaction with FcγRIIB Meanwhile, the present disclosure also provides a method for screening and obtaining these mutants. As shown in FIG. 2, Fc mutants with specific affinity properties can be screened according to the above method.

Furthermore, screen amino acid mutations that enhance various protein-protein interactions or protein-other molecules interactions according to the above screening method provided by the present disclosure; a general screening method includes:

1) providing a parent protein sequence, introducing amino acid mutations in the parent protein sequence by PCR;

2) constructing the parent protein and the mutated protein into an expression vector to form a mutation library;

3) transfecting the expression vector into mammalian cells, and expressing the parent protein and the mutated protein on the cell surface;

4) incubating a labeled interacting protein and the mammalian cell, and selecting labeled mammalian cells by flow cytometry or magnetic-activated cell sorting;

5) extracting DNAs from the labeled mammalian cells, sequencing the DNAs, and analyzing the change of the sequence proportions before and after sorting; and, the amino acid mutation in which the proportion after sorting is significantly higher than the proportion before sorting is an amino acid mutation capable of enhancing the binding affinity between the protein and the interacting protein.

### Example 1: Construction of Human Antibody IgG2_Fc Point Mutation Library

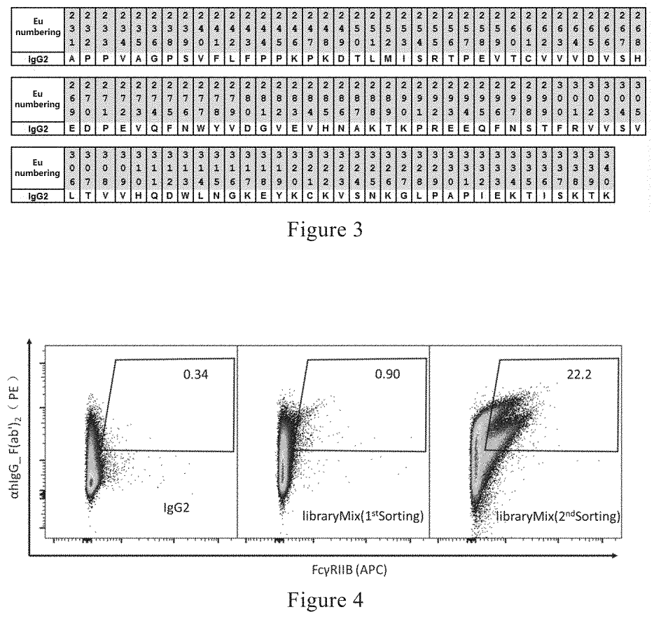

Some sites on human IgG2 Fc CH2 are selected, including P233-S239, V266-P271, 5298-T299 and G327-I332 (Eu numbering, see FIGS. 3 and 4). Amino acids on these sites are randomly mutated, and each site includes 20 types of amino acids (A, R, D, C, Q, E, H, I, G, N, L, K, M, F, P, S, T, W, Y, V) in total, including wild-type. Single-point random mutation plasmid library corresponding to the above four regions of human IgG2 Fc respectively (Library1, Library2, Library3, Library4), and a mixture library of these four libraries (LibraryMix) were constructed.

### Example 2: Cell Enrichment after Flow Cytometry Sorting Relative to Before Sorting

After the plasmid library is successfully constructed, retrovirus is prepared to infect 3T3 cells, to obtain the cell lines stably expressing the libraries. The present disclosure uses flow cytometry sorting techniques to sort cells expressing human IgG2 (detected by anti-human IgG F(ab′)2) and binding human FcγRIIB FIG. 4 shows that flow cytometry sorting can gradually enrich cells with high binding affinity to FcγRIIB The cells obtained by flow cytometry sorting for different rounds of human IgG2 library LibraryMix are respectively analyzed. The flow cytometry analysis shows that the proportion of the cells binding to FcγRIIB is increased after each round. The proportion after the first round is 0.90% and that of the second round is 22.2%, which increased 2.65 times and 65.29 times, respectively, as compared to 0.34% of wild-type human IgG2.

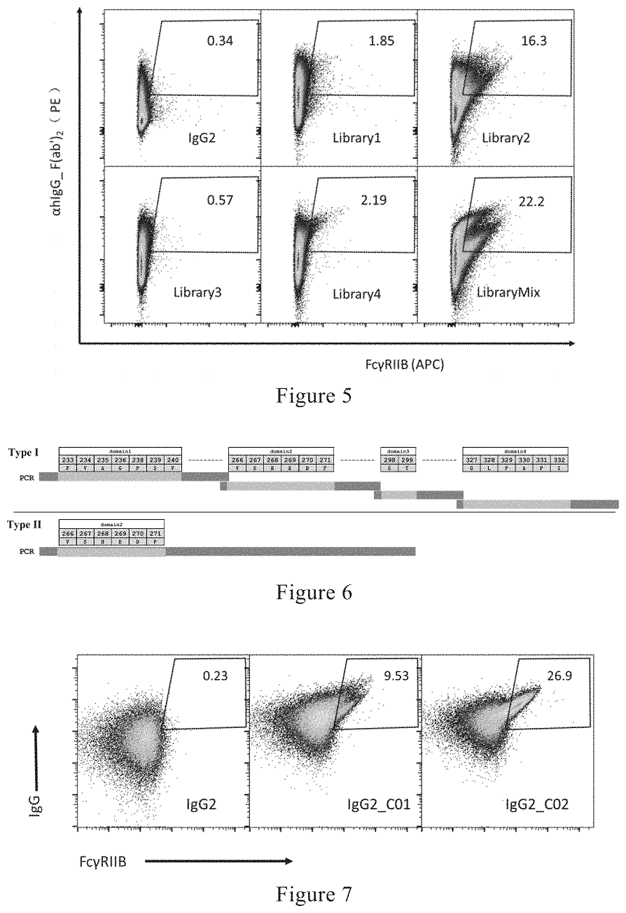

As shown in FIG. 5, after two rounds of sorting, the proportion of the FcγRIIB-binding cells of Library1, Library2, Library3, Library4 and LibraryMix detected by flow cytometry are increased to 1.85%, 16.3%, 0.57%, 2.19% and 22.2%, respectively, which are 5.44 times, 47.94 times, 1.69 times, 6.44 times and 65.29 times higher than 0.34% of wild type human IgG2.

### Example 3: NGS Analysis of IgG2 Fc Mutation Types with Enhanced FcγRIIB Binding Affinity after Flow Cytometry Sorting

Mutations with the ratio of the proportion of sequence after sorting to the proportion before sorting increased to be greater than twice that of wild-type human IgG2 (enrichment factor/wild-type enrichment factor >2) are selected, i.e., mutations on human IgG2 which is beneficial to the improvement of the binding affinity of IgG2 to FcγRIIB (at least twice that of the wild-type enrichment factor), see Table 7. For example, the proportion of L328W before sorting is 0.39%, and the proportion after sorting is 12.84%, therefore the enrichment factor is 33.20, which is 22.21 times the wild-type enrichment factor (1.49).

TABLE 7
Mutations in human IgG2 which is beneficial to the
improvement of the binding affinity of IgG2 to FcγRIIB

| | | | | | | Enrichment | |
|---|---|---|---|---|---|---|---|
| | | | | | | factor | Increased |
| | | | | | | Percentage | factor |
| | | | | | | after | Enrichment |
| | | | | | | sorting/ | factor/ |
| | | Before sorting | | After sorting | | percentage | wild-type |
| | | | Per- | | Per- | before | enrichment |
| NO. | Mutation | count | centage | Count | centage | sorting | factor |

Library2

| | | | | | | | |
|---|---|---|---|---|---|---|---|
| #1 | Wild-type | 12137 | 11.34 | 7266 | 6.51 | 0.57 | 1.00 |
| 1 | S267Q | 410 | 0.38 | 3693 | 3.31 | 8.64 | 15.05 |
| 2 | S267A | 744 | 0.70 | 2457 | 2.20 | 3.17 | 5.52 |
| 3 | S267D | 513 | 0.48 | 1128 | 1.01 | 2.11 | 3.67 |
| 4 | S267M | 794 | 0.74 | 1223 | 1.10 | 1.48 | 2.57 |
| 5 | S267G | 10 | 0.00 | 27 | 0.01 | 1.79 | 3.50 |
| 6 | H268E | 319 | 0.30 | 561 | 0.50 | 1.69 | 2.94 |
| 7 | H268D | 221 | 0.21 | 355 | 0.32 | 1.54 | 2.68 |
| 8 | H268K | 421 | 0.39 | 581 | 0.52 | 1.32 | 2.31 |
| 9 | P271C | 893 | 0.83 | 15969 | 14.30 | 17.14 | 29.87 |
| 10 | V266A/P271C | 5 | 0.00 | 220 | 0.20 | 42.18 | 73.50 |
| 11 | V266A/P271G | 3 | 0.00 | 27 | 0.02 | 8.63 | 15.03 |
| 12 | S267A/P271C | 3 | 0.00 | 117 | 0.10 | 37.39 | 65.14 |
| 13 | S267A/P271G | 2 | 0.00 | 11 | 0.01 | 5.27 | 9.19 |
| 14 | S267E/D270H | 2 | 0.00 | 89 | 0.08 | 42.66 | 74.33 |
| 15 | S267E/P271C | 2 | 0.00 | 2089 | 1.87 | 1001.34 | 1744.71 |
| 16 | S267E/P271V | 2 | 0.00 | 46 | 0.04 | 22.05 | 38.42 |
| 17 | S267E/P271W | 2 | 0.00 | 50 | 0.04 | 23.97 | 41.76 |
| 18 | S267E/P271Y | 4 | 0.00 | 80 | 0.07 | 19.17 | 33.41 |
| 19 | S267M/P271C | 2 | 0.00 | 67 | 0.06 | 32.12 | 55.96 |
| 20 | S267M/P271G | 2 | 0.00 | 9 | 0.01 | 4.31 | 7.52 |
| 21 | V266G/P271C | 3 | 0.00 | 12 | 0.01 | 3.83 | 6.68 |

Library4

| | | | | | | | |
|---|---|---|---|---|---|---|---|
| #2 | wild-type | 14727 | 16.91 | 17343 | 25.27 | 1.49 | 1.00 |
| 22 | G327A | 713 | 0.82 | 2838 | 4.13 | 5.05 | 3.38 |
| 23 | L328F | 3 | 0.00 | 1332 | 1.94 | 563.47 | 377.03 |
| 24 | L328W | 337 | 0.39 | 8815 | 12.84 | 33.20 | 22.21 |
| 25 | L328Y | 50 | 0.06 | 133 | 0.19 | 3.38 | 2.26 |
| 26 | G327A/A330R | 2 | 0.00 | 155 | 0.23 | 98.35 | 65.81 |
| 27 | G327A/A330V | 2 | 0.00 | 11 | 0.02 | 6.98 | 4.67 |
| 28 | G327A/I332A | 2 | 0.00 | 46 | 0.07 | 29.19 | 19.53 |
| 29 | G327A/I332C | 1 | 0.00 | 9 | 0.01 | 11.42 | 7.64 |
| 30 | G327A/I332E | 1 | 0.00 | 99 | 0.14 | 125.64 | 84.07 |
| 31 | L328E/I332T | 10 | 0.01 | 434 | 0.63 | 55.08 | 36.85 |

LibraryMix

| | | | | | | | |
|---|---|---|---|---|---|---|---|
| #3 | wild-type | 91423 | 29.03 | 119849 | 21.72 | 0.75 | 1.00 |
| 32 | S267E | 259 | 0.08 | 98580 | 17.86 | 217.23 | 290.34 |
| 33 | S267R | 6 | 0.00 | 16 | 0.00 | 1.52 | 2.03 |
| 34 | P271G | 1132 | 0.36 | 136659 | 24.77 | 68.90 | 92.09 |
| 35 | P271Q | 218 | 0.07 | 631 | 0.11 | 1.65 | 2.21 |
| 36 | S298L | 2696 | 0.86 | 10298 | 1.87 | 2.18 | 2.91 |
| 37 | L328M | 14 | 0.00 | 611 | 0.11 | 24.91 | 33.29 |
| 38 | S267V/S298L | 9 | 0.00 | 33 | 0.01 | 2.09 | 2.80 |
| 39 | S267E/S298R | 7 | 0.00 | 402 | 0.07 | 32.78 | 43.81 |
| 40 | P271A/S298R | 7 | 0.00 | 30 | 0.01 | 2.45 | 3.27 |
| 41 | P271G/G236V | 6 | 0.00 | 108 | 0.02 | 10.27 | 13.73 |
| 42 | P271G/P329S | 7 | 0.00 | 567 | 0.10 | 46.23 | 61.79 |
| 43 | P271G/P331C | 7 | 0.00 | 1717 | 0.31 | 139.99 | 187.11 |
| 44 | P271G/P331T | 12 | 0.00 | 680 | 0.12 | 32.34 | 43.23 |
| 45 | P271G/S298D | 8 | 0.00 | 95 | 0.02 | 6.78 | 9.06 |
| 46 | P271G/S298E | 10 | 0.00 | 5218 | 0.95 | 297.81 | 398.04 |
| 47 | P271G/S298G | 20 | 0.01 | 341 | 0.06 | 9.73 | 13.01 |
| 48 | P271G/S298K | 6 | 0.00 | 107 | 0.02 | 10.18 | 13.60 |
| 49 | P271G/S298L | 13 | 0.00 | 4286 | 0.78 | 188.17 | 251.50 |
| 50 | P271G/S298N | 8 | 0.00 | 252 | 0.05 | 17.98 | 24.03 |
| 51 | P271G/S298R | 18 | 0.01 | 504 | 0.09 | 15.98 | 21.36 |
| 52 | P271G/T299A | 23 | 0.01 | 253 | 0.05 | 6.28 | 8.39 |
| 53 | P271G/T299M | 10 | 0.00 | 83 | 0.02 | 4.74 | 6.33 |
| 54 | P271G/T299S | 6 | 0.00 | 126 | 0.02 | 11.99 | 16.02 |
| 55 | P271G/T299W | 6 | 0.00 | 97 | 0.02 | 9.23 | 12.33 |

#1-3: Data from the experiments of Library 2 (#1), Library 4 (#2), LibraryMix (#3).

In addition to single point mutation, some double mutations were also obtained, and the ratios of these amino acid mutations after flow cytometry sorting to that before sorting are increased by more than twice as compared to the wild-type human IgG2. The double mutation combination (V266A/P271C or G, S267A/P271C or G, S267E/D270H, S267E/P271C or V or W or Y, S267E/S298R, S267M/P271C or G, P271A/S298R, P271G/G236V, P271G/G329S, P271G/P331C or T, P271G/S298D or E or G or K or L or N or R, P271G/T299A or M or S or W, G327A/A330R or V, G327A/I332A or C or E, L328E/I332T, etc.) has a higher increased factor (ratio) than any point mutation thereof, indicating that both amino acids in the mutation combination contribute to the increase in the binding affinity to FcγRIIB In addition, mutation combinations such as S267V/S298L provide an increased factor approximately equal to S298L mutation, which has a higher increased factor over S267V, indicating that the improvement of the mutation combination is basically only the contribution of S298L, rather than the other point mutation, as shown in Table 8. Therefore, in addition to the amino acid mutation provided by the present disclosure, the Fc region, the antibody, the Fc fragment or the fusion protein of the present disclosure may further include other possible combinations or modifications.

TABLE 8
Double mutations on human IgG2 which is beneficial to the
improvement of the binding affinity of IgG2 to FcγRIIB

| | | Enrichment | | | | | |
|---|---|---|---|---|---|---|---|
| | | factor | Increased | | | | |
| | | Percentage | factor ^{a} | | | | |
| | | after | Enrichment | | | | |
| | | sorting/ | factor/ | | | | |
| | | percentage | wild-type | Increased | | Increased | |
| | | before | enrichment | factor ^{b} | | factor 2 ^{c} | |
| NO. | Mutation | sorting | factor | Site 1 | Site 2 | Site 1 | Site 2 |
| 1 | V266A/P271C | 42.18 | 73.5 | 3.1 | 29.9 | 24.1 | 2.5 |
| 2 | V266A/P271G | 8.63 | 15.03 | 3.1 | 4.7 | 4.9 | 3.2 |
| 3 | S267A/P271C | 37.39 | 65.14 | 5.5 | 29.9 | 11.8 | 2.2 |
| 4 | S267A/P271G | 5.27 | 9.19 | 5.5 | 4.7 | 1.7 | 1.9 |
| 5 | S267E/D270H | 42.66 | 74.33 | 28.5 | 1.3 | 2.6 | 56.7 |
| 6 | S267E/P271C | 1001.34 | 1744.71 | 28.5 | 29.9 | 61.2 | 58.4 |
| 7 | S267E/P271V | 22.05 | 38.42 | 28.5 | 0.7 | 1.4 | 52.6 |
| 8 | S267E/P271W | 23.97 | 41.76 | 28.5 | 0.4 | 1.5 | 101.9 |
| 9 | S267E/P271Y | 19.17 | 33.41 | 28.5 | 1.0 | 1.2 | 33.4 |
| 10 | S267E/S298R | 32.78 | 43.81 | 28.5 | 0.8 | 1.5 | 56.2 |
| 12 | S267M/P271C | 32.12 | 55.96 | 2.6 | 29.9 | 21.8 | 1.9 |
| 13 | S267M/P271G | 4.31 | 7.52 | 2.6 | 4.7 | 2.9 | 1.6 |
| 14 | P271A/S298R | 2.45 | 3.27 | 0.1 | 0.8 | 54.5 | 4.2 |
| 15 | P271G/G236V | 10.27 | 13.73 | 4.7 | 0.5 | 2.9 | 30.5 |
| 16 | P271G/P329S | 46.23 | 61.79 | 4.7 | 1.2 | 13.1 | 52.8 |
| 17 | P271G/P331C | 139.99 | 187.11 | 4.7 | 0.2 | 39.6 | 779.6 |
| 18 | P271G/P331T | 32.34 | 43.23 | 4.7 | 0.1 | 9.1 | 480.3 |
| 19 | P271G/S298D | 6.78 | 9.06 | 4.7 | 1.6 | 1.9 | 5.5 |
| 20 | P271G/S298E | 297.81 | 398.04 | 4.7 | 7.1 | 84.2 | 56.0 |
| 21 | P271G/S298G | 9.73 | 13.01 | 4.7 | 1.2 | 2.8 | 10.7 |
| 22 | P271G/S298K | 10.18 | 13.6 | 4.7 | 4.4 | 2.9 | 3.1 |
| 23 | P271G/S298L | 188.17 | 251.5 | 4.7 | 2.9 | 53.2 | 86.4 |
| 24 | P271G/S298N | 17.98 | 24.03 | 4.7 | 2.3 | 5.1 | 10.5 |
| 25 | P271G/S298R | 15.98 | 21.36 | 4.7 | 0.8 | 4.5 | 27.4 |
| 26 | P271G/T299A | 6.28 | 8.39 | 4.7 | 0.4 | 1.8 | 20.5 |
| 27 | P271G/T299M | 4.74 | 6.33 | 4.7 | 1.0 | 1.3 | 6.1 |
| 28 | P271G/T299S | 11.99 | 16.02 | 4.7 | 0.6 | 3.4 | 25.0 |
| 29 | P271G/T299W | 9.23 | 12.33 | 4.7 | 0.5 | 2.6 | 23.3 |
| 30 | G327A/A330R | 98.35 | 65.81 | 3.4 | 0.0 | 19.5 | 21936.7 |
| 31 | G327A/A330V | 6.98 | 4.67 | 3.4 | 1.0 | 1.4 | 4.8 |
| 32 | G327A/I332A | 29.19 | 19.53 | 3.4 | 16.4 | 5.8 | 1.2 |
| 33 | G327A/I332C | 11.42 | 7.64 | 3.4 | 2.7 | 2.3 | 2.9 |
| 34 | G327A/I332E | 125.64 | 84.07 | 3.4 | 1.1 | 24.9 | 79.3 |
| 35 | L328E/I332T | 55.08 | 36.85 | 0.1 | 1.6 | 335.0 | 22.6 |

^{a} Ratio of mutant enrichment factor/wild-type enrichment factor after sorting.
^{b} Ratio of mutant enrichment factor/wild-type enrichment factor after sorting regarding the original point mutation.
^{c} Ratio of increase factor of combined mutation/increase factor of one point mutation.

### Example 4: Analysis of Favorable Mutant Types by NGS after Magnetic-Activated Sorting

The high-FcγRIIB-affinity cells obtained from the above flow cytometry sorting are further sorted by magnetic-activated sorting. The cells are incubated with the mixture of Biotin-labeled FcγRIIB and non-labeled activating FcγRs, and then the labeled cells are sorted by anti-Biotin magnetic beads. The enrichment ratios after sorting relative to before sorting are analyzed by NGS. Mutations with a higher I/A ratio can be screened, as shown in Table 9. Mutations are selected according to the following criteria: the ratio of the mutation after sorting to that before sorting is increased by more than twice as compared to the wild-type human IgG2, and the proportion of the mutation is decreased in the sorting portion not binding to magnetic bead (i.e., the outflow liquid passing the LS column).

TABLE 9
Mutations in human IgG2 leading to the improvement of the binding
affinity of human IgG2 to FcγRIIB, and showing lower binding affinity of human
IgG2 to the activating FcγRs than that to FcγRIIB

| | | | | | | | | | | Enrichment | Increased |
|---|---|---|---|---|---|---|---|---|---|---|---|
| | | | | | | | | Comparison before | | factor | factor |
| | | | | | | | | and after sorting | | Percentage | Mutation |
| | | | | Portion not | | | | Percentage | Percentage | after | enrichment |
| | | | | binding to | | | | after | before | sorting/ | factor/ |
| | | Before sorting | | magnetic bead | | After sorting | | sorting/ | sorting/ | percentage | wild-type |
| | | | Percen- | | Percen- | | Percen- | unbinding | unbinding | before | enrichment |
| NO. | Mutation | Count | tage | Count | tage | Amount | tage | percentage | percentage | sorting | factor |

Library2

| | | | | | | | | | | | |
|---|---|---|---|---|---|---|---|---|---|---|---|
| #1 | Wild- | 2838 | 3.60 | 5139 | 5.15 | 2509 | 2.98 | 0.58 | 0.70 | 0.83 | 1.00 |
| | type | | | | | | | | | | |
| 1 | S267N | 66 | 0.08 | NA | NA | 205 | 0.24 | — | — | 2.91 | 3.51 |
| 2 | S267W | 89 | 0.11 | 4 | 0 | 177 | 0.21 | 52.49 | 28.16 | 1.86 | 2.25 |
| 3 | H268N | 226 | 0.29 | 1 | 0 | 558 | 0.66 | 661.97 | 286.03 | 2.31 | 2.79 |
| 4 | P271A | 61 | 0.08 | 3 | 0 | 180 | 0.21 | 71.18 | 25.73 | 2.77 | 3.33 |
| 5 | P271I | 311 | 0.39 | NA | NA | 873 | 1.04 | — | — | 2.63 | 3.17 |
| 6 | P271L | 51 | 0.06 | 2 | 0 | 241 | 0.29 | 142.95 | 32.27 | 4.43 | 5.34 |
| 7 | P271S | 64 | 0.08 | 10 | 0.01 | 187 | 0.22 | 22.18 | 8.1 | 2.74 | 3.3 |
| 8 | P271V | 246 | 0.31 | 1 | 0 | 923 | 1.1 | 1094.98 | 311.35 | 3.52 | 4.24 |

Library4

| | | | | | | | | | | | |
|---|---|---|---|---|---|---|---|---|---|---|---|
| #2 | Wild- | 11937 | 14.33 | 16838 | 14.78 | 13554 | 12.62 | 0.85 | 0.97 | 0.88 | 1.00 |
| | type | | | | | | | | | | |
| 9 | G327L | 14 | 0.02 | 19 | 0.02 | 68 | 0.06 | 3.8 | 1.01 | 3.77 | 4.28 |
| 10 | G327S | 83 | 0.1 | 11 | 0.01 | 738 | 0.69 | 71.16 | 10.32 | 6.9 | 7.84 |
| 11 | L328N | 109 | 0.13 | 8 | 0.01 | 384 | 0.36 | 50.91 | 18.63 | 2.73 | 3.11 |

LibraryMix

| | | | | | | | | | | | |
|---|---|---|---|---|---|---|---|---|---|---|---|
| #3 | Wild- | 14919 | 19.10 | 20509 | 20.31 | 21855 | 21.53 | 1.06 | 0.94 | 1.13 | 1.00 |
| | type | | | | | | | | | | |
| 12 | L328G | 12 | 0.02 | NA | NA | 47 | 0.05 | — | — | 3.01 | 2.67 |
| 13 | L328R | 18 | 0.02 | 4 | 0 | 124 | 0.12 | 30.84 | 5.82 | 5.3 | 4.69 |
| 14 | L328V | 17 | 0.02 | 2 | 0 | 51 | 0.05 | 25.37 | 10.99 | 2.31 | 2.04 |
| 15 | P271G/ | 8 | 0.01 | 2 | 0 | 24 | 0.02 | 11.94 | 5.17 | 2.31 | 2.04 |
| | S298N | | | | | | | | | | |
| 16 | H268S | 30 | 0.04 | NA | NA | 306 | 0.3 | — | — | 7.85 | 6.95 |
| #4 | Wild- | 14919 | 19.10 | 20860 | 18.89 | 18101 | 17.95 | 0.95 | 1.01 | 0.94 | 1.00 |
| | type | | | | | | | | | | |
| 17 | P238Q/ | 40 | 0.05 | 48 | 0.04 | 191 | 0.19 | 4.36 | 1.18 | 3.7 | 3.93 |
| | S267E | | | | | | | | | | |
| 18 | S267E/ | 27 | 0.03 | 30 | 0.03 | 169 | 0.17 | 6.17 | 1.27 | 4.85 | 5.16 |
| | P329R | | | | | | | | | | |
| 19 | P271G/ | 28 | 0.04 | 7 | 0.01 | 102 | 0.1 | 15.96 | 5.66 | 2.82 | 3 |
| | I332L | | | | | | | | | | |
| 20 | S267E | 13 | 0.02 | 1 | 0 | 48 | 0.05 | 52.56 | 18.38 | 2.86 | 3.04 |
| 21 | P271G | 11 | 0.01 | 3 | 0 | 52 | 0.05 | 18.98 | 5.18 | 3.66 | 3.89 |
| 22 | L328W | 18 | 0.02 | NA | NA | 99 | 0.1 | — | — | 4.26 | 4.53 |
| 23 | L328A | 28 | 0.04 | NA | NA | 116 | 0.12 | — | — | 3.21 | 3.41 |
| 24 | L328P | 29 | 0.04 | 3 | 0 | 81 | 0.08 | 29.57 | 13.67 | 2.16 | 2.3 |

NA: In the sample of the column, the mutation of the row is not detected.
#1-#4: Data from Library 2 (#1), Library 4 (#2), and two experiments of LibraryMix (#3, #4).

The affinity of human IgG1 to FcγRIIB can be increased by G236D, S239D (Seung Y Chu et al. Molecular Immunology 45 (2008) 3926-3933) and S298A mutation (US20090042291A1). However, in the results of NGS analysis of the present disclosure, the proportion of the IgG2 variant comprising G236D or S298A after sorting is not increased, but decreased (Table 10); the proportion of the human IgG2 variant containing S239D before sorting is 0.026%, and is not detected after sorting. These results demonstrate that G236D, S239D, and S298A mutations do not increase the FcγRIIB binding affinity of human IgG2, see Table 10.

TABLE 10
The effect of the known mutation on the binding affinity of IgG2
to FcγR cannot be directly predicted according to the effect
of the mutation on the binding affinity of IgG1 to FcγR.

| | | | | | | Enrichment |
|---|---|---|---|---|---|---|
| | | | | | | factor |
| | | Before sorting | | After sorting | | Percentage after |
| | | | Percen- | | Percen- | sorting/percentage |
| NO. | Mutant | Count | tage | Count | tage | before sorting |
| 1 | S298A | 4221 | 4.05 | 4205 | 3.72 | 0.92 |
| 2 | S239D | 29 | 0.03 | 0 | NA | — |
| 3 | G236D | 511 | 0.46 | 427 | 0.39 | 0.84 |

NA: The mutant of the row is not detected in the experiment of the column, and the percentage and factor cannot be calculated.

### Example 5: Amino Acid Mutations in Human IgG2 Fc Polypeptide Fragments which can Enhance the Affinity to FcγRIIB

Table 11 summarizes the amino acid mutations capable of enhancing affinity to FcγRIIB screened by the point mutation library screening method according to the present disclosure. Compared with patents in the prior art, it has been found that the sites S267G and P271C of the present disclosure are both new mutation types on human IgG2 that can improve the binding affinity to FcγRIIB The sites H268S, L328A and G327A are all new mutation types on human IgG2 that improve the binding affinity to FcγRIIB and show lower affinity to other activating FcγRs than that to FcγRIIB

TABLE 11
Amino acid mutations in human IgG2
that enhance the affinity to FcγRIIB

| Mutation | | | | | | | | | | | | |
|---|---|---|---|---|---|---|---|---|---|---|---|---|
| site | Original | | | | | | | | | | | |
| (Eu num- | amino | Amino acid mutation | | | | | | | | | | |
| bering) | acid | enhancing affinity to FcγRIIB | | | | | | | | | | |
| 267 | S | E | Q | A | D | M | G | R | V | N | W | |
| 268 | H | E | D | K | N | S | | | | | | |
| 271 | P | C | G | Q | V | W | Y | A | I | L | S | |
| 327 | G | A | L | S | | | | | | | | |
| 328 | L | F | M | W | Y | E | A | G | N | P | R | V |

### Example 6: Construction of Human Antibody IgG2_Fc Combined Mutation Library

According to the Fc mutation type capable of increasing the affinity to FcγRIIB (the mutation having a higher enrichment factor by more than 1 time over wild-type) screened by the above point mutation libraries and the high-throughput sequencing results, two types of combined mutation library were designed. Type I: combined point mutations among four regions, i.e., regions P233-V240, V266-P271, S298-T299 and G327-I332 each have 0-1 mutation, and the combined mutation contains 0-4 point mutations. Type II: combined mutation of multiple amino acids in one region, i.e., regions P233-V240, V266-P271, S298-T299 or G327-I332 has combined mutation within each region (respectively 0-7, 0-6, 0-2 and 0-6 combined mutations), as shown in FIG. 6. The size of the plasmid library is up to 4.67*10⁵.

### Example 7: Cell Enrichment Before and After Flow Cytometry Sorting for Combined Mutation Library

According to point mutation screening and high-throughput sequencing results, Type I and Type II combined mutation libraries are constructed (Example 6), and the cell libraries expressing the combined mutation are obtained. Then flow cytometry is used for sorting the cells with high affinity to FcγRIIB for two to three rounds. As shown in FIG. 7, after two rounds of sorting, the proportion of the cells of library IgG2_C01 and library IgG2_C02 detected by flow cytometry that are capable of binding to FcγRIIB are increased to 9.53% and 26.9%, respectively, which are 41.43 times and 116.96 times higher than 0.23% of wild type human IgG2. IgG2_C01 is a combined mutation (Type I) library having at most 1 mutation in each of the four regions of P233-V240, V266-P271, S298-T299, and G327-I332; IgG2_C02 is a cell library made by combining the combined mutation (Type II) libraries of four regions of P233-V240, V266-P271, S298-T299 and G327-I332 together and transferring it into 3T3 cells.

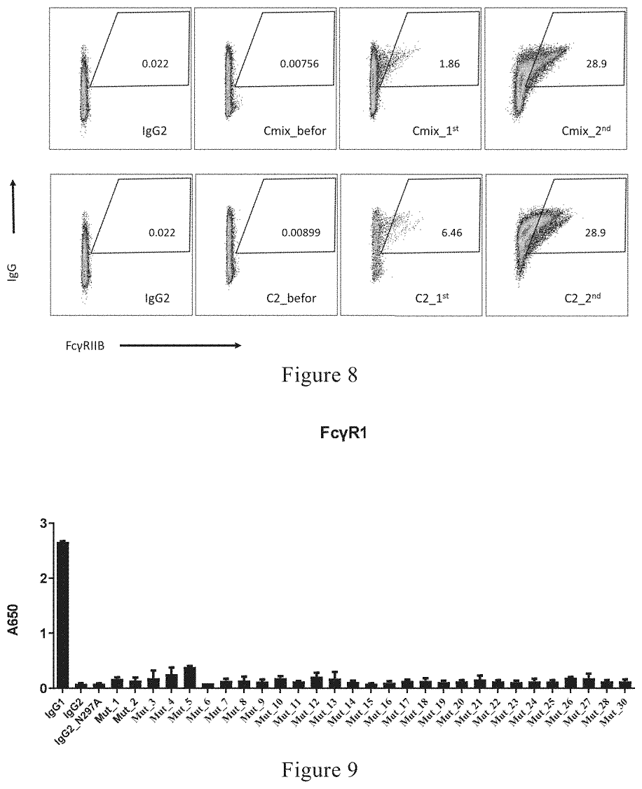

As shown in FIG. 8, after two rounds of sorting, the binding affinity of the cells of library Cmix and library C2 cells to FcγRIIB is increased significantly, according to flow cytometry. The proportion of the cells of the libraries that are capable of binding to FcγRIIB is increased upon each round of sorting: Cmix shows a cell proportion of 1.86% and 28.9% that are capable of binding to FcγRIIB after the first round of sorting and the second round of sorting, respectively, which are 246.03 times and 3822.75 times higher than 0.00756% before sorting; C2 reaches 6.46% and 28.9%, which are 718.58 times and 3214.68 times higher than 0.00899% before sorting. The library Cmix is a combined mutation (Type I) library with at most one mutation in each of the four regions; and the library C2 is a combined mutation (Type II) library of the V266-P271 region.

### Example 8: NGS Analysis of IgG2 Fc Combined Mutation Showing Enhanced FcγRIIB Binding Affinity after Flow Cytometry Sorting

In order to screen the IgG2 Fc combined mutation type with increased binding affinity to FcγRIIB, the combined mutant library constructed in the present disclosure is subjected to flow cytometry sorting to obtain cells with higher binding affinity to FcγRIIB, then the enrichment factor of each mutation type after sorting relative to before sorting is analyzed by NGS. The result is shown in Table 12. The screened mutation has a higher enrichment factor (after the last round of sorting/before sorting), or shows a significant enrichment in at least one round of sorting (after said round of sorting/before said round of sorting), or is only detected after the last round of sorting. For example, the enrichment factor of V266L/S298L/L328W is 21920.33 times of the wild type (after the last round of sorting/before sorting); the enrichment factor of the point mutation L328W is 8.78 times of the wild type (after the last round of sorting/before sorting); the enrichment factors of H268D/S298L/L328W are 43.98 (after the second round of sorting/before the second round of sorting) and 5.95 (after the third round of sorting/before the third round of sorting); the enrichment factor of V234M/S267E/S298L/L328W is 5.13 (after third round sorting/before third round sorting); V234Q/A235G/P238L/S239V/G327A/L328E/A330S/I332T is only detected after the last round of sorting.

TABLE 12
Mutations on human IgG2 screened from the combined mutation library,
which are beneficial to the improvement of the binding affinity of IgG2 to FcγRIIB

| | | | | | Enrichment factor | | | |
|---|---|---|---|---|---|---|---|---|
| | | | | | | | | Increased |
| | | | | | | | | factor |
| | | | | | Percentage | Percentage | Percentage | Mutant |
| | | | | | after 2nd | after 1st | after 2nd | enrichment |
| | | Percentage | | | sorting/ | sorting/ | sorting/ | factor/ |
| | | After | After | | percentage | percentage | percentage | wild-type |
| | | 2nd | 1st | Before | after | before | before | enrichment |
| NO. | | sorting | sorting | sorting | 1st sorting | sorting | sorting | factor |
| #1 | WT | 54.22 | 61.56 | 63.83 | 0.88 | 0.96 | 0.85 | 1.00 |
| 1 | L328W | 1.05 | 0.25 | 0.14 | 4.26 | 1.75 | 7.46 | 8.78 |
| 2 | S267E/A330S | 0.03 | 0.00 | NA | 43.11 | — | — | — |
| 3 | S267E/S298G | 0.25 | 0.01 | 0.00 | 19.16 | 9.16 | 175.56 | 206.66 |
| 4 | P233G/S267E | 0.05 | 0.01 | 0.00 | 5.47 | 3.31 | 18.10 | 21.31 |
| 5 | P233G/S267E/S298G | 0.20 | 0.01 | 0.00 | 38.49 | 7.13 | 274.31 | 322.90 |
| #2 | WT | 47.81 | 58.39 | 56.60 | 0.82 | 1.03 | 0.84 | 1.00 |
| 6 | V266L/S267E/ | 0.25 | NA | NA | — | — | — | — |
| | H268S/P271V | | | | | | | |
| 7 | V234Q/A235G/ | 0.15 | NA | NA | — | — | — | — |
| | P238L/S239V/ | | | | | | | |
| | G327A/L328E/ | | | | | | | |
| | A330S/I332T | | | | | | | |
| 8 | V234Q/A235G/ | 0.07 | NA | NA | — | — | — | — |
| | P238L/S239V/ | | | | | | | |
| | S267E/H268S/ | | | | | | | |
| | E269D | | | | | | | |
| 9 | S267E/H268S/ | 5.78 | 0.07 | 0.00 | 83.65 | 80.49 | 6733.01 | 7971.36 |
| | E269D | | | | | | | |

| | | | | | | Percentage | Percentage | Percentage | Percentage | Mutant |
|---|---|---|---|---|---|---|---|---|---|---|
| | | | | | | after 3rd | after 2nd | after 1st | after 3rd | enrichment/ |
| | | | | | | sorting/ | sorting/ | sorting/ | sorting/ | factor |
| | | After | After | After | | percentage | percentage | percentage | percentage | wild-type |
| | | 3rd | 2nd | 1st | Before | after 2nd | after 1st | before | before | enrichment |
| | | sorting | sorting | sorting | sorting | sorting | sorting | sorting | sorting | factor |
| #3 | WT | 0.40 | 1.03 | 1.32 | 1.07 | 0.39 | 0.78 | 1.24 | 0.38 | 1.00 |
| 10 | H268D/S298L/ | 1.25 | 0.21 | 0.00 | NA | 5.95 | 43.98 | — | — | — |
| | L328W | | | | | | | | | |
| 11 | V266L/L328W | 1.20 | 0.22 | 0.00 | 0.00 | 5.53 | 53.28 | 4.71 | 1386.84 | 3695.56 |
| 12 | V266L/S298L/ | 24.98 | 4.14 | 0.06 | 0.00 | 6.03 | 64.77 | 21.07 | 8226.06 | 21920.33 |
| | L328W | | | | | | | | | |
| 13 | S267V/S298L/ | 0.90 | 0.13 | 0.00 | 0.00 | 7.21 | 73.61 | 3.92 | 2081.88 | 5547.67 |
| | L328W | | | | | | | | | |
| 14 | S239V/V266L/ | 0.33 | 0.07 | 0.00 | NA | 5.02 | 39.07 | — | — | — |
| | S298L/L328W | | | | | | | | | |
| 15 | A235W/V266L/ | 0.22 | 0.05 | 0.00 | NA | 4.67 | 70.06 | — | — | — |
| | S298L/L328W | | | | | | | | | |
| 16 | P233F/V266L/ | 0.29 | 0.16 | 0.00 | NA | 1.79 | 94.73 | — | — | — |
| | S298L/L328W | | | | | | | | | |
| 17 | S267V/S298G/ | 0.19 | 0.03 | 0.00 | NA | 5.76 | 47.36 | — | — | — |
| | L328W | | | | | | | | | |
| 18 | V234M/S267E/ | 0.53 | 0.10 | NA | NA | 5.13 | — | — | — | — |
| | S298L/L328W | | | | | | | | | |
| 19 | V234M/S267E/ | 0.30 | 0.46 | 0.01 | 0.00 | 0.66 | 64.37 | 16.47 | 695.04 | 1852.11 |
| | S298G/I332L | | | | | | | | | |
| #4 | WT | 12.58 | 5.25 | 2.51 | 8.97 | 2.40 | 2.09 | 0.28 | 1.40 | 1.00 |
| 20 | V266L/S267E/H | 6.76 | 0.95 | 0.25 | 0.00 | 7.11 | 3.84 | 60.52 | 1651.90 | 1178.72 |
| | 268S/E269A/P271C | | | | | | | | | |
| 21 | V266L/S267E/ | 4.15 | 2.13 | 0.17 | 0.00 | 1.95 | 12.85 | 99.90 | 2501.18 | 1784.72 |
| | H268S/E269V | | | | | | | | | |
| 22 | V266L/S267E/ | 0.11 | 0.01 | 0.00 | NA | 12.64 | 6.97 | — | — | — |
| | H268S/E269V/ | | | | | | | | | |
| | P271C | | | | | | | | | |
| 23 | V266L/S267E/ | 1.23 | 0.24 | 0.03 | 0.00 | 5.20 | 9.34 | 32.69 | 1588.00 | 1133.12 |
| | H268S/E269G/ | | | | | | | | | |
| | P271T | | | | | | | | | |
| 24 | V266L/S267D/ | 1.88 | 0.49 | 0.12 | 0.00 | 3.80 | 4.07 | 121.76 | 1882.93 | 1343.57 |
| | H268D/E269D/ | | | | | | | | | |
| | P271Q | | | | | | | | | |
| 25 | V266L/S267E/ | 1.22 | 0.42 | 0.14 | 0.00 | 2.94 | 2.92 | 214.29 | 1838.25 | 1311.69 |
| | E269K/P271G | | | | | | | | | |
| 26 | V234Q/V266L/ | 0.04 | 0.00 | NA | NA | 15.39 | — | — | — | — |
| | S267D/H268D/ | | | | | | | | | |
| | E269D/P271Q | | | | | | | | | |
| 27 | V234Q/V266L/ | 0.09 | 0.01 | NA | NA | 11.37 | — | — | — | — |
| | S267E/H268S/ | | | | | | | | | |
| | E269V | | | | | | | | | |
| 28 | V266L/S267M/ | 13.35 | 17.07 | 2.59 | 0.01 | 0.78 | 6.60 | 229.11 | 1182.52 | 843.79 |
| | H268E/P271Q | | | | | | | | | |

#1-4: Data from the experiments of Libraries IgG2_C01 (#1), IgG2_C02 (#2), Cmix (#3), C2 (#4).
NA: In the sample of the column, the mutation of the row is not detected.

### Example 9: The Binding Property of Mutant Antibodies to FcγR Analyzed by Enzyme-Linked Immunosorbent Assay (ELISA)

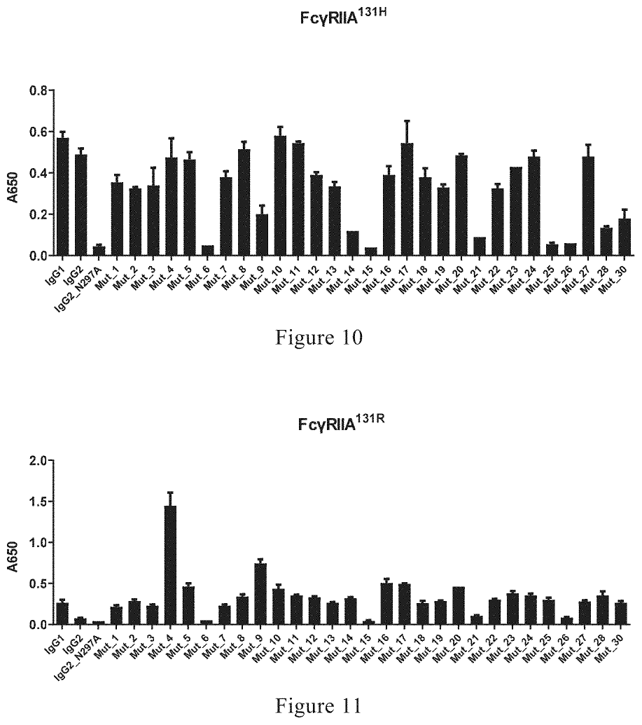

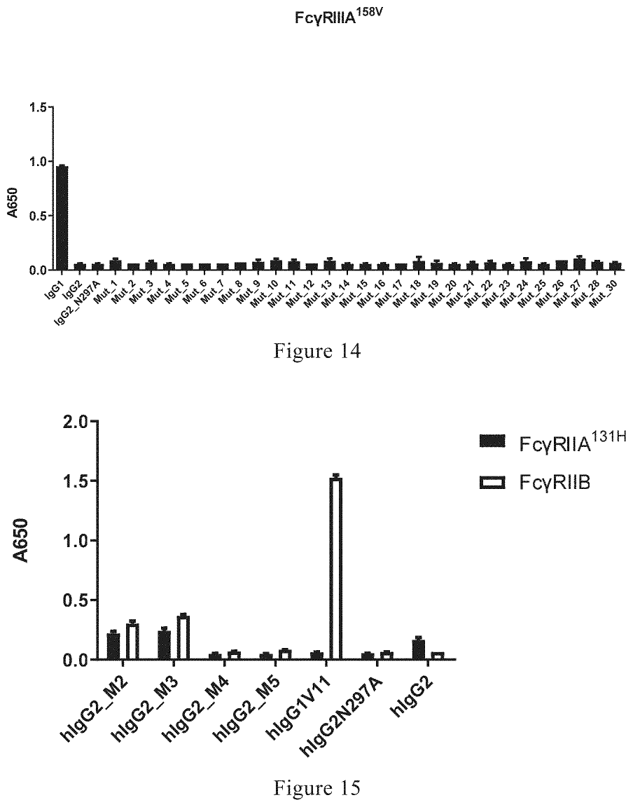

On the basis of the favored Fc single-point mutation and combined mutation screened above by NGS, new Fc mutation combinations are constructed. Anti-human OX40 antibodies containing these new Fc mutation combinations are expressed and the binding property of the mutant antibody to FcγR are analyzed by Enzyme-linked immunosorbent assay (ELISA). FIGS. 9-14 show the binding affinity of different Fc mutants to FcγRI, FcγRIIA^{131H}, FcγRIIA^{131R}, FcγRIIB, FcγRIIIA^{158F} and FcγRIIIA^{158V}. By designating the binding affinity of IgG1 to each FcγR to 1, the relative binding affinity of each mutant to the FcγR (the ELISA absorbance value of the binding of the mutant to the FcγR divided by the ELISA absorbance value of the binding of IgG1 to the FcγR) and the increased factor of the binding affinity of each mutant to FcγRIIB as compared with that of wild type IgG2 (the ELISA absorbance value of the binding of the mutant to FcγRIIB divided by the ELISA absorbance value of the binding of wild-type IgG2 to FcγRIIB) are calculated. As shown in Table 13, the increased factor of the binding affinity of each mutant to FcγRIIB as compared with that of wild-type IgG2 is 4.23 times to 25.55 times. In addition, the ratio of the binding affinity of each mutant to FcγRIIB to the binding affinity of said mutant to FcγRIIA′ (RIIB/RIIAR) is calculated, and the results (Table 13) show that the RIIB/RIIAR ratio of these mutants is higher than that of wild-type IgG2 (wild-type IgG2: 0.34; mutants: 0.46-3.67).

TABLE 13
Binding of different IgG2 antibody Fc mutants to FcγR.

| | | | | | | | Relative binding affinity | | | | | | | |
|---|---|---|---|---|---|---|---|---|---|---|---|---|---|---|
| | | | | | | | | | | | Increased | | | |
| | | | | | | | | | | | factor | | | |
| | | | | | | | | | | | of | | | |
| | | | | | | | | | | | RIIB | | | |
| | | | | | | | | | | | binding | | | |
| | | | | | | | | | | | com- | | | |
| | | | | | | | | | | | pared | | | |
| | | | | | | | | | | | with | | | |
| | | | | | | | | | | | wild- | | | |
| | Absorbance value | | | | | | RI/ | RIIAH/ | RIIAR/ | RIIB/ | type | RIIIAF/ | RIIIAV/ | RIIB/ |
| | RI | RIIAH | RIIAR | RIIB | RIIIAF | RIIIAV | G1 | G1 | G1 | G1 | IgG2 | G1 | G1 | RIIAR |
| IgG1 | 2.66 | 0.57 | 0.26 | 0.76 | 0.19 | 0.96 | | | | | | | | |
| IgG2 | 0.08 | 0.49 | 0.08 | 0.08 | 0.06 | 0.05 | 0.03 | 0.85 | 0.29 | 0.10 | 1.00 | 0.30 | 0.06 | 0.34 |
| IgG2 | 0.08 | 0.04 | 0.04 | 0.07 | 0.06 | 0.06 | 0.03 | 0.08 | 0.16 | 0.09 | 0.93 | 0.31 | 0.06 | 0.57 |
| N297A | | | | | | | | | | | | | | |
| Mut 1 | 0.17 | 0.35 | 0.22 | 0.64 | 0.07 | 0.09 | 0.06 | 0.62 | 0.83 | 0.85 | 8.51 | 0.38 | 0.09 | 1.02 |
| Mut 2 | 0.14 | 0.32 | 0.29 | 1.00 | 0.07 | 0.06 | 0.05 | 0.56 | 1.09 | 1.33 | 13.38 | 0.38 | 0.06 | 1.21 |
| Mut 3 | 0.18 | 0.34 | 0.23 | 0.94 | 0.08 | 0.07 | 0.07 | 0.60 | 0.87 | 1.25 | 12.57 | 0.40 | 0.08 | 1.44 |
| Mut 4 | 0.25 | 0.48 | 1.44 | 1.92 | 0.06 | 0.05 | 0.10 | 0.84 | 5.51 | 2.54 | 25.55 | 0.32 | 0.06 | 0.46 |
| Mut 5 | 0.38 | 0.46 | 0.46 | 1.18 | 0.06 | 0.06 | 0.14 | 0.81 | 1.77 | 1.56 | 15.69 | 0.31 | 0.07 | 0.88 |
| Mut 6 | 0.09 | 0.05 | 0.05 | 0.50 | 0.06 | 0.06 | 0.03 | 0.08 | 0.19 | 0.66 | 6.63 | 0.30 | 0.06 | 3.52 |
| Mut 7 | 0.13 | 0.38 | 0.23 | 0.58 | 0.07 | 0.06 | 0.05 | 0.67 | 0.87 | 0.76 | 7.70 | 0.38 | 0.07 | 0.88 |
| Mut 8 | 0.14 | 0.52 | 0.34 | 1.07 | 0.08 | 0.07 | 0.05 | 0.90 | 1.30 | 1.42 | 14.30 | 0.44 | 0.07 | 1.09 |
| Mut 9 | 0.12 | 0.20 | 0.74 | 1.20 | 0.07 | 0.07 | 0.05 | 0.34 | 2.83 | 1.59 | 15.99 | 0.38 | 0.08 | 0.56 |
| Mut_10 | 0.18 | 0.58 | 0.43 | 1.05 | 0.09 | 0.09 | 0.07 | 1.02 | 1.64 | 1.40 | 14.06 | 0.48 | 0.09 | 0.85 |
| Mut_11 | 0.13 | 0.55 | 0.36 | 1.01 | 0.08 | 0.08 | 0.05 | 0.96 | 1.36 | 1.33 | 13.40 | 0.41 | 0.08 | 0.98 |
| Mut_12 | 0.21 | 0.39 | 0.33 | 0.94 | 0.06 | 0.06 | 0.08 | 0.68 | 1.26 | 1.25 | 12.57 | 0.32 | 0.06 | 0.99 |
| Mut_13 | 0.18 | 0.33 | 0.26 | 0.73 | 0.07 | 0.08 | 0.07 | 0.58 | 1.01 | 0.97 | 9.75 | 0.37 | 0.09 | 0.96 |
| Mut 14 | 0.12 | 0.12 | 0.32 | 0.70 | 0.06 | 0.05 | 0.04 | 0.21 | 1.22 | 0.93 | 9.36 | 0.31 | 0.06 | 0.76 |
| Mut_15 | 0.08 | 0.04 | 0.04 | 0.47 | 0.06 | 0.05 | 0.03 | 0.07 | 0.17 | 0.62 | 6.21 | 0.31 | 0.06 | 3.67 |
| Mut_16 | 0.10 | 0.39 | 0.50 | 1.09 | 0.06 | 0.06 | 0.04 | 0.68 | 1.92 | 1.44 | 14.51 | 0.32 | 0.06 | 0.75 |
| Mut_17 | 0.14 | 0.54 | 0.50 | 1.19 | 0.09 | 0.06 | 0.05 | 0.95 | 1.90 | 1.58 | 15.93 | 0.49 | 0.06 | 0.83 |
| Mut_18 | 0.14 | 0.38 | 0.26 | 0.78 | 0.09 | 0.08 | 0.05 | 0.67 | 1.00 | 1.04 | 10.43 | 0.47 | 0.09 | 1.03 |
| Mut_19 | 0.11 | 0.33 | 0.28 | 0.82 | 0.07 | 0.07 | 0.04 | 0.58 | 1.08 | 1.09 | 10.96 | 0.38 | 0.07 | 1.00 |
| Mut_20 | 0.12 | 0.48 | 0.46 | 1.13 | 0.06 | 0.05 | 0.05 | 0.85 | 1.76 | 1.50 | 15.08 | 0.31 | 0.06 | 0.85 |
| Mut_21 | 0.16 | 0.09 | 0.11 | 0.32 | 0.06 | 0.06 | 0.06 | 0.16 | 0.41 | 0.42 | 4.23 | 0.31 | 0.06 | 1.03 |
| Mut_22 | 0.12 | 0.33 | 0.31 | 0.93 | 0.06 | 0.07 | 0.05 | 0.57 | 1.17 | 1.22 | 12.33 | 0.33 | 0.07 | 1.05 |
| Mut_23 | 0.11 | 0.43 | 0.38 | 0.97 | 0.07 | 0.06 | 0.04 | 0.75 | 1.44 | 1.29 | 12.96 | 0.35 | 0.06 | 0.90 |
| Mut_24 | 0.12 | 0.48 | 0.35 | 1.00 | 0.08 | 0.08 | 0.05 | 0.84 | 1.35 | 1.32 | 13.33 | 0.42 | 0.08 | 0.98 |
| Mut_25 | 0.12 | 0.06 | 0.30 | 0.86 | 0.07 | 0.05 | 0.05 | 0.10 | 1.13 | 1.14 | 11.49 | 0.36 | 0.06 | 1.01 |
| Mut_26 | 0.19 | 0.06 | 0.08 | 0.55 | 0.08 | 0.09 | 0.07 | 0.11 | 0.32 | 0.72 | 7.28 | 0.41 | 0.09 | 2.24 |
| Mut_27 | 0.18 | 0.48 | 0.28 | 0.70 | 0.09 | 0.10 | 0.07 | 0.84 | 1.06 | 0.92 | 9.29 | 0.49 | 0.11 | 0.87 |
| Mut_28 | 0.13 | 0.14 | 0.35 | 0.88 | 0.08 | 0.08 | 0.05 | 0.24 | 1.35 | 1.17 | 11.76 | 0.44 | 0.08 | 0.87 |
| Mut_30 | 0.12 | 0.18 | 0.26 | 0.80 | 0.06 | 0.06 | 0.05 | 0.32 | 1.01 | 1.06 | 10.65 | 0.33 | 0.07 | 1.05 |

Note:
RI, RIIAH, RIIAR, RIIB, RIIIAF, RIIIAV respectively represent FcγRI, FcγRIIA^{131H}, FcγRIIA^{131R}, FcγRIIB, FcγRIIIA^{158F}, FcγRIIIA^{158V}.

### Example 10: Beneficial Mutations Support Stronger Immune Activation Activity

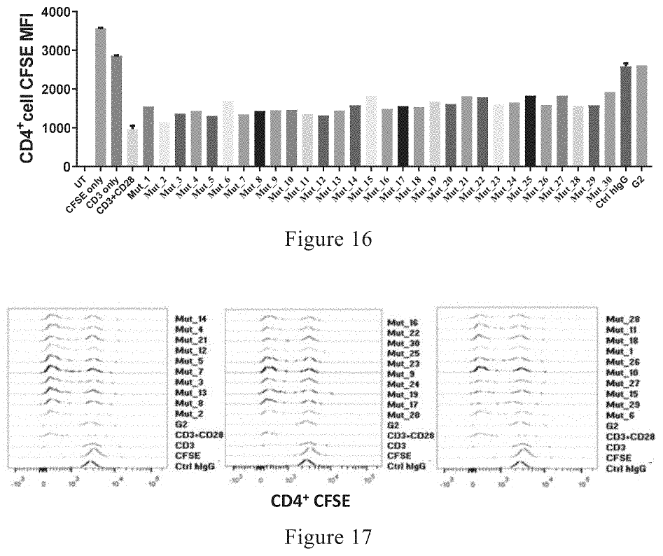

The immune activation activity of the anti-human OX40 antibody (Example 9) containing the beneficial mutation is analyzed in the FCγR/OX40 humanized mouse spleen cells. FIGS. 16 and 17 show the CFSE fluorescence intensity and the corresponding histogram of CD4⁺ cells when the antibody concentration is 1 μg/mL, where low CFSE fluorescence intensity reflects high activity, and the activity of all the selected antibodies is better than that of the wild-type IgG2. The activity values of different antibody concentrations of the two experiments are analyzed together. The activity of the group with αCD3 antibody only was set as 0, while the activity of the positive control group with αCD3 antibody and αCD28 antibody was set as 1. The relative activity of each mutant antibody is calculated, and the average value is calculated. Table 14 shows that the activity of the selected 30 mutant antibodies is superior to that of the wild type IgG2, and the activity is increased to 9.17 to 16.51 times of the wild type IgG2.

TABLE 14
Immunostimulatory activity analysis of anti-human
OX40 antibodies containing different Fc mutations

| | | | | | | | | Experiment 2 | | | | | | |
|---|---|---|---|---|---|---|---|---|---|---|---|---|---|---|
| | | | | | | | | | | | | | | Average |
| | | | | | | | | | | | | | | increased |
| | | | | | | | | | | | | | | factor |
| | Experiment 1 | | | | | | | | | | | | | of the |
| | CFSE | | | | | | | CFSE | | | | | | activity |
| | fluorescence | | | | | | | fluorescence | | Relative | | | Average | over |
| | intensity | | | Relative activity | | | | intensity | | activity | | | relative | wild-type |
| | 1 | 0.1 | 0.01 | 1 | 0.1 | 0.01 | Average | 1 | 0.1 | 1 | 0.1 | Average | activity | IgG2 |
| | μg/ | μg/ | μg/ | μg/ | μg/ | μg/ | relative | μg/ | μg/ | μg/ | μg/ | relative | of two | of two |
| | ml | ml | ml | ml | ml | ml | activity | ml | ml | ml | ml | activity | experiments | experiments |
| CD3 + CD28 | 509 | 473 | 491 | 0.97 | 1.03 | 1.00 | 1.00 | 965 | 965 | 1.00 | 1.00 | 1.00 | 1.00 | |
| Mut_1 | 799 | 786 | 894 | 0.44 | 0.47 | 0.27 | 0.39 | 1552 | 2062 | 0.69 | 0.42 | 0.56 | 0.46 | 10.48 |
| Mut_2 | 677 | 654 | 685 | 0.66 | 0.71 | 0.65 | 0.67 | 1149 | 1548 | 0.90 | 0.69 | 0.80 | 0.72 | 16.51 |
| Mut_3 | 686 | 568 | 761 | 0.65 | 0.86 | 0.51 | 0.67 | 1365 | 1769 | 0.79 | 0.58 | 0.68 | 0.68 | 15.47 |
| Mut_4 | 769 | 616 | 695 | 0.50 | 0.77 | 0.63 | 0.63 | 1439 | 1702 | 0.75 | 0.61 | 0.68 | 0.65 | 14.91 |
| Mut_5 | 791 | 695 | 859 | 0.46 | 0.63 | 0.33 | 0.47 | 1309 | 1660 | 0.82 | 0.63 | 0.73 | 0.58 | 13.14 |
| Mut_6 | 706 | 689 | 766 | 0.61 | 0.64 | 0.50 | 0.59 | 1698 | 1986 | 0.61 | 0.46 | 0.54 | 0.57 | 12.94 |
| Mut_7 | 741 | 755 | 850 | 0.55 | 0.52 | 0.35 | 0.47 | 1347 | 1819 | 0.80 | 0.55 | 0.67 | 0.55 | 12.66 |
| Mut_8 | 799 | 719 | 844 | 0.44 | 0.59 | 0.36 | 0.46 | 1436 | 1813 | 0.75 | 0.55 | 0.65 | 0.54 | 12.32 |
| Mut_9 | 744 | 742 | 849 | 0.54 | 0.55 | 0.35 | 0.48 | 1454 | 1909 | 0.74 | 0.50 | 0.62 | 0.54 | 12.27 |
| Mut_10 | 765 | 743 | 788 | 0.50 | 0.54 | 0.46 | 0.50 | 1465 | 2048 | 0.74 | 0.43 | 0.58 | 0.54 | 12.23 |
| Mut_11 | 783 | 831 | 782 | 0.47 | 0.39 | 0.47 | 0.44 | 1359 | 1868 | 0.79 | 0.52 | 0.66 | 0.53 | 12.10 |
| Mut_12 | 762 | 689 | 906 | 0.51 | 0.64 | 0.25 | 0.47 | 1319 | 2085 | 0.81 | 0.41 | 0.61 | 0.52 | 11.99 |
| Mut_13 | 802 | 728 | 899 | 0.44 | 0.57 | 0.26 | 0.42 | 1446 | 1865 | 0.75 | 0.53 | 0.64 | 0.51 | 11.62 |
| Mut_14 | 793 | 775 | 839 | 0.45 | 0.49 | 0.37 | 0.44 | 1581 | 1899 | 0.68 | 0.51 | 0.59 | 0.50 | 11.40 |
| Mut_15 | 691 | 707 | 837 | 0.64 | 0.61 | 0.37 | 0.54 | 1826 | 2253 | 0.55 | 0.32 | 0.43 | 0.50 | 11.37 |
| Mut_16 | 783 | 770 | 853 | 0.47 | 0.50 | 0.35 | 0.44 | 1487 | 2158 | 0.72 | 0.37 | 0.55 | 0.48 | 11.01 |
| Mut_17 | 775 | 788 | 841 | 0.49 | 0.46 | 0.37 | 0.44 | 1557 | 2120 | 0.69 | 0.39 | 0.54 | 0.48 | 10.95 |
| Mut_18 | 776 | 828 | 838 | 0.48 | 0.39 | 0.37 | 0.42 | 1535 | 2135 | 0.70 | 0.38 | 0.54 | 0.47 | 10.65 |
| Mut_19 | 824 | 735 | 880 | 0.40 | 0.56 | 0.30 | 0.42 | 1672 | 2089 | 0.63 | 0.41 | 0.52 | 0.46 | 10.46 |
| Mut_20 | 774 | 844 | 828 | 0.49 | 0.36 | 0.39 | 0.41 | 1610 | 2161 | 0.66 | 0.37 | 0.51 | 0.45 | 10.37 |
| Mut_21 | 782 | 711 | 920 | 0.47 | 0.60 | 0.22 | 0.43 | 1814 | 2194 | 0.55 | 0.35 | 0.45 | 0.44 | 10.08 |
| Mut_22 | NA | NA | NA | — | — | — | — | 1791 | 2290 | 0.56 | 0.30 | 0.43 | 0.43 | 9.90 |
| Mut_23 | 843 | 777 | 898 | 0.36 | 0.48 | 0.26 | 0.37 | 1600 | 2161 | 0.67 | 0.37 | 0.52 | 0.43 | 9.80 |
| Mut_24 | 816 | 812 | 912 | 0.41 | 0.42 | 0.24 | 0.36 | 1654 | 2052 | 0.64 | 0.43 | 0.53 | 0.43 | 9.75 |
| Mut_25 | 779 | 778 | 895 | 0.48 | 0.48 | 0.27 | 0.41 | 1827 | 2209 | 0.55 | 0.34 | 0.45 | 0.42 | 9.69 |
| Mut_26 | 753 | 806 | 938 | 0.53 | 0.43 | 0.19 | 0.38 | 1591 | 2368 | 0.67 | 0.26 | 0.47 | 0.42 | 9.50 |
| Mut_27 | 834 | 807 | 849 | 0.38 | 0.43 | 0.35 | 0.39 | 1827 | 2214 | 0.55 | 0.34 | 0.44 | 0.41 | 9.36 |
| Mut_28 | 862 | 882 | 829 | 0.33 | 0.29 | 0.39 | 0.34 | 1566 | 2230 | 0.68 | 0.33 | 0.51 | 0.41 | 9.26 |
| Mut_29 | NA | NA | NA | — | — | — | — | 1581 | 2611 | 0.68 | 0.13 | 0.40 | 0.40 | 9.23 |
| Mut_30 | NA | NA | NA | — | — | — | — | 1925 | 2277 | 0.49 | 0.31 | 0.40 | 0.40 | 9.17 |
| WTIgG2 | 1060 | 1016 | 1002 | −0.03 | 0.05 | 0.08 | 0.03 | 2610 | 2885 | 0.13 | -0.01 | 0.06 | 0.04 | 1.00 |
| Ctrl hIgG | 1071 | 1083 | 1018 | −0.05 | −0.07 | 0.05 | −0.02 | 2587 | 2730 | 0.15 | 0.07 | 0.11 | 0.03 | 0.65 |
| CD3 0nly | 1051 | 1037 | 1044 | −0.01 | 0.01 | 0.00 | 0.00 | 2863 | 2863 | 0.00 | 0.00 | 0.00 | 0.00 | 0.00 |

NA: Mutant antibody of the row was not subjected to the experiment of the column.

### Example 11: The Effect of the Known Mutation on the Binding Ability of IgG2 to FcγR Cannot be Directly Predicted According to the Effect of the Mutation on the Binding Ability of IgG1 to FcγR

The affinity of human IgG1 to FcγRIIB can be increased by G236D, S239D (Seung Y Chu et al. Molecular Immunology 45 (2008) 3926-3933) and S298A mutation (US20090042291A1). However, in the results of NGS analysis of the present disclosure, the proportion of the IgG2 variant comprising G236D or S298A after sorting is not increased, but decreased (Table 10); the proportion of the human IgG2 variant containing S239D before sorting is 0.026%, and is not detected after sorting. These results demonstrate that G236D, S239D, and S298A mutations do not increase the FcγRIIB binding capacity of human IgG2, see Table 10.

In addition, we also introduce into IgG2 the V11 mutations (G237D/P238D/H268D/P271G/A330R) capable of significantly improving the binding affinity of IgG1 to FcγRIIB (F Mimoto et al., Protein Engineering, Design & Selection vol. 26 no. 10 pp. 589-598, 2013) to obtain IgG2_M2(H268D/P271G), IgG2_M3(H268D/P271G/A330R), IgG2_M4 (G236D/P238D/H268D/P271G) and IgG2_M5(G236D/P238D/H268D/P271G/A330R), and the mutations of M5 in IgG2 corresponds to the mutations of IgG1 V11. ELISA results of FIG. 15 show that these mutants do not provide IgG2 with the same binding affinity to FcγRIIB as IgG1 V11.

Therefore, the effect of the known mutation on the binding ability of IgG2 to FcγR cannot be directly predicted according to the effect of the mutation on the binding ability of IgG1 to FcγR.

Each reference provided herein is incorporated by reference to the same extent as if each reference was individually incorporated by reference. In addition, it should be understood that based on the above teaching content of the disclosure, various changes or modifications to the disclosure, and these equivalent forms also fall within the scope of the appended claims.

1. A mutant Fc polypeptide fragment, wherein the mutant Fc polypeptide fragment has the following features:
(i) said mutant Fc polypeptide fragment has a mutation at position(s) selected from the group consisting of: L328, H268, S267, P271, G327, or a combination thereof, with respect to the wild-type Fc polypeptide fragment of SEQ ID NO: 20, wherein the numbering of all amino acids is based on the IgG Eu numbering; and
(ii) the affinity of the mutant Fc polypeptide fragment to FcγRIIB is increased compared to the wild-type Fc polypeptide fragment of SEQ ID NO: 20.

2. A mutant immunoglobulin Fc region, wherein the mutant immunoglobulin Fc region comprises the mutant Fc polypeptide fragment according to claim 1.

3. An antibody, wherein the antibody comprises the mutant Fc polypeptide fragment according to claim 1 or a mutant immunoglobulin Fc region comprising the mutant Fc polypeptide fragment.

4. A fusion protein, wherein the fusion protein comprises the mutant Fc polypeptide fragment according to claim 1, a mutant immunoglobulin Fc region or an antibody comprising the mutant Fc polypeptide fragment.

5. An isolated polynucleotide, wherein the polynucleotide encodes the mutant Fc polypeptide fragment according to claim 1, or encodes a mutant immunoglobulin Fc region, a antibody, or a fusion protein comprising the mutant Fc polypeptide fragment.

6. A vector, wherein the vector comprises the isolated polynucleotide according to claim 5.

7. A host cell, wherein
the host cell expresses the mutant Fc polypeptide fragment according to claim 1, or expresses a mutant immunoglobulin Fc region, a antibody, or a fusion protein comprising the mutant Fc polypeptide fragment.

8. A pharmaceutical composition, wherein the pharmaceutical composition comprises:
(a) the mutant Fc polypeptide fragment according to claim 1, or a mutant immunoglobulin Fc region, a antibody, or a fusion protein comprising the mutant Fc polypeptide fragment; and
(b) a pharmaceutically acceptable carrier.

9. A method of producing a mutant Fc polypeptide fragment, or a mutant immunoglobulin Fc region, an antibody, or a fusion protein comprising the mutant Fc polypeptide fragment,
wherein the mutant Fc polypeptide fragment has the following features:
(i) said mutant Fc polypeptide fragment has a mutation at position(s) selected from the group consisting of: L328, H268, S267, P271, G327, or a combination thereof, with respect to the wild-type Fc polypeptide fragment of SEQ ID NO: 20, wherein the numbering of all amino acids is based on the IgG Eu numbering; and
(ii) the affinity of the mutant Fc polypeptide fragment to FcγRIIB is increased compared to the wild-type Fc polypeptide fragment of SEQ ID NO: 20,
wherein the method comprises the steps of:
(i) culturing the host cell according to claim 7 under suitable conditions to obtain a culture comprising said mutant Fc polypeptide fragment, said mutant immunoglobulin Fc region, said antibody, or said fusion protein; and
(ii) purifying and/or separating said mutant Fc polypeptide fragment, said mutant immunoglobulin Fc region, said antibody, or said fusion protein from the culture obtained in step (i).

10. (canceled)

11. A method of treating a disease, comprising the steps of: administering to a subject in need thereof:
the mutant Fc polypeptide fragment according to claim 1;
a mutant immunoglobulin Fc region, an antibody, or a fusion protein comprising the mutant Fc polypeptide fragment;
an isolated polynucleotide encoding the mutant Fc polypeptide fragment, the mutant immunoglobulin Fc region, the antibody, or the fusion protein;
a vector comprising the isolated polynucleotide; or
a host cell comprising the vector.

12. A method of treating a disease, comprising the steps of: administering to a subject in need thereof the pharmaceutical composition according to claim 8.

13. The method according to claim 12, wherein the subject comprises a mammal.

14. The method according to claim 12, wherein the disease is selected from the group consisting of: cancer, inflammatory diseases, autoimmune diseases, or combinations thereof.

15. The method according to claim 14, wherein the cancer includes: breast cancer, prostate cancer, colon cancer, throat cancer, brain cancer, blood cancer; basal cell carcinoma, biliary cancer, bladder cancer, bone cancer, brain and central nervous system cancer, cervical cancer, chorionic carcinoma, colorectal cancer, connective tissue cancer, digestive system cancer, endometrial cancer, esophageal cancer, ocular cancer, head and neck cancer, gastric cancer, epithelial neoplasm, renal cancer, throat cancer, liver cancer, lung cancer, lymphoma; melanoma; neuroblastoma; oral cancer; ovarian cancer; pancreatic cancer; retinoblastoma; rhabdomyosarcoma; rectal cancer; respiratory cancer; sarcoma; skin cancer; gastric cancer; testicular cancer; thyroid cancer; uterine cancer; urinary system cancer; and other cancers and sarcoma.

16. The mutant Fc polypeptide fragment according to claim 1, wherein,
the mutation at L328 is selected form the group consisting of: L328W, L328E, L328Y, L328F, L328M, L328A, L328G, L328N, L328P, L328R, and L328V;
the mutation at H268 is selected form the group consisting of: H268D, H268S, H268E, H268K, and H268N;
the mutation at 5267 is selected form the group consisting of: S267E, S267V, S267D, S267M, S267Q, S267A, S267G, S267R, S267N, and S267W;
the mutation at P271 is selected form the group consisting of: P271C, P271G, P271V, P271A, P271W, P271Y, P271Q, P271T, P271I, P271L, and P271S;
the mutation at G327 is selected form the group consisting of: G327A, G327L, and G327S.

17. The mutant Fc polypeptide fragment according to claim 16, wherein,
the mutation at L328 is L328W or L328E;
the mutation at H268 is H268D, H268S, or H268E;
the mutation at 5267 is S267E, S267V, S267D, or S267M;
the mutation at P271 is P271C, P271G, P271V, P271A, P271W, P271Y, P271Q, or P271T;
the mutation at G327 is G327A.

18. The mutant Fc polypeptide fragment according to claim 1, wherein, the mutant Fc polypeptide fragment has a mutation selected from the group consisting of L328W, L328E, H268D, H268S, H268E, S267V, S267E, A330S, P233G, P271G, P271C, P271V, P271A, P271W, P271Y, and G327A, or a combination thereof, with respect to the wild-type Fc fragment; wherein the numbering of all amino acids is based on the IgG Eu numbering.

19. The mutant Fc polypeptide fragment according to claim 1, wherein,
the mutant Fc polypeptide fragment has a mutation of L328W or L328E, and optionally has a mutation at one or more positions selected from the group consisting of: H268, S267, P271, and G327, with respect to the wild-type Fc fragment;
the mutant Fc polypeptide fragment has a mutation of H268D, H268S or H268E, and optionally has a mutation at one or more positions selected from the group consisting of: L328, S267, P271, and G327, with respect to the wild-type Fc fragment;
the mutant Fc polypeptide fragment has a mutation of S267V, S267D or S267M, and optionally has a mutation at one or more positions selected from the group consisting of: L328, H268, P271, and G327, with respect to the wild-type Fc fragment;
the mutant Fc polypeptide fragment has a mutation of P271C, P271G, P271V, P271A, P271W, P271Y, P271Q or P271T, and optionally has a mutation at one or more positions selected from the group consisting of: L328, H268, 5267, and G327, with respect to the wild-type Fc fragment; or
the mutant Fc polypeptide fragment has a mutation of G327A, and optionally has a mutation at one or more positions selected from the group consisting of: L328, H268, S267, and P271, with respect to the wild-type Fc fragment.

20. The mutant Fc polypeptide fragment according to claim 1, wherein, the mutant Fc polypeptide fragment has a mutation in at least one of L328 and H268 with respect to the wild-type Fc fragment, and the specific mutations in the mutant Fc polypeptide fragment are selected from one of the group consisting of: L328W, L328E, H268D, H268S, H268E, H268D/S298L/L328W, S267V/S298L/L328W, V234M/S267E/S298L/L328W, A235W/V266L/S298L/L328W, V234Q/A235G/P238L/S239V/H268D/G327A/L328E/A330S/I332T, S239V/V266L/S298L/L328W, V266L/L328W, V266L/S267D/H268D/E269D/P271Q, S267E/H268 S/E269D, P233F/V266L/S298L/L328W, V266L/S298L/L328W, V234Q/A235G/P238L/S239V/G327A/L328E/A330S/I332T, V266L/S267E/H268S/E269V, V266L/S267E/H268S/E269V/P271C, V266L/S267E/H268S/E269A/P271C, S267V/S298G/L328W, V266L/S267M/H268E/P271Q, V266L/S267E/H268S/E269G/P271T, V234Q/V266L/S267D/H268D/E269D/P271Q, V234Q/A235G/P238L/S239V/S267E/H268S/E269D, V266L/S267E/H268S/P271V, V234Q/A235G/P238L/S239V/S267E/S298L/G327A/L328E/A330S/I332T, V234Q/V266L/S267E/H268S/E269V, and L328E/I332T; wherein the numbering of all amino acids is based on the IgG Eu numbering; or
the mutant Fc polypeptide fragment has a mutation at S267 with respect to the wild-type Fc fragment, and the specific mutation in the mutant Fc polypeptide fragment is selected from one of the group consisting of: S267E/A330S, S267E/S298G, P233G/S267E, V234M/S267E/S298G/I332L, P233 G/S267E/S298G, V266L/S267E/E269K/P271G, P238Q/S267E, S267A/P271C, S267A/P271G, S267E/D270H, S267E/P271C, S267E/P271V, S267E/P271W, S267E/P271Y, S267E/S298R, S267M/P271C, S267M/P271G, S267V/S298L, and S267E/P329R; wherein the numbering of all amino acids is based on the IgG Eu numbering; or
the mutant Fc polypeptide fragment has a mutation at P271 with respect to the wild-type Fc fragment, and the specific mutation in the mutant Fc polypeptide fragment is selected from one of the group consisting of: V266L/S267E/E269K/P271G, V266A/P271C, V266A/P271G, S267A/P271C, S267A/P271G, S267E/P271C, S267E/P271V, S267E/P271W, S267E/P271Y, S267M/P271C, S267M/P271G, V266G/P271C, P271A/S298R, P271G/G236V, P271G/P329S, P271G/P331C, P271G/P331T, P271G/S298D, P271G/S298E, P271G/S298G, P271G/S298K, P271G/S298L, P271G/S298N, P271G/S298R, P271G/T299A, P271G/T299M, P271G/T299S, P271G/T299W, and P271G/I332L; wherein the numbering of all amino acids is based on the IgG Eu numbering; or
the mutant Fc polypeptide fragment has a mutation at G327 with respect to the wild-type Fc fragment, and the specific mutation in the mutant Fc polypeptide fragment is selected from one of the group consisting of: G327A/A330R, G327A/A330V, G327A/I332A, G327A/I332C, and G327A/I332E; wherein the numbering of all amino acids is based on the IgG Eu numbering.

21. The mutant Fc polypeptide fragment according to claim 1, wherein, the specific mutation of the mutant Fc polypeptide fragment with respect to the wild-type Fc fragment is selected from one of the group consisting of: H268D/S298L/L328W, S267V/S298L/L328W, V234M/S267E/S298L/L328W, A235W/V266L/S298L/L328W, and V234Q/A235G/P238L/S239V/H268D/G327A/L328E/A330S/I332T; wherein the numbering of all amino acids is based on the IgG Eu numbering.
